(19) United States
(12) Patent Application Publication
(10) Pub. No.: US 2013/0224268 A1
(43) Pub. Date: Aug. 29, 2013

(54) TOPICAL DELIVERY OF HORMONAL AND NON HORMONAL NANO FORMULATIONS, METHODS OF MAKING AND USING THE SAME

(71) Applicant: NEWGEN BIOPHARMA CORP., (US)

(72) Inventors: Abu ALAM, Lake Forest, IL (US); Navdeep JAIKARIA, Titusville, NJ (US)

(73) Assignee: NEWGEN BIOPHARMA CORP., Titusville, NJ (US)

(21) Appl. No.: 13/778,954

(22) Filed: Feb. 27, 2013

Related U.S. Application Data
(60) Provisional application No. 61/603,814, filed on Feb. 27, 2012.

(51) Int. Cl. A61K 8/04; A61K 45/06

(52) U.S. Cl. CPC A61K 8/04; A61K 45/06
USPC 424/401; 424/400; 514/170; 514/182

(57) ABSTRACT

The present invention relates to methods for treating, preventing, minimizing, and/or diminishing signs of aging in the skin comprising topically administering to the subject in need thereof a hormonal nano formulation.

### CROSS REFERENCE TO RELATED APPLICATIONS

This application claims priority of U.S. Provisional Patent Application No. 61/603,814, filed on Feb. 27, 2012, the contents of which are specifically incorporated herein by reference.

### FIELD OF THE INVENTION

The present invention relates to hormonal formulations and methods of use, where the nano formulation comprises at least one active agent which is a hormone. Additional non-hormonal active agents can also be incorporated into the composition. The compositions are useful for administration for treating and/or preventing any therapeutic indication associated with the active agent, such as wrinkles and signs of aging skin for hormonal active agents. The method comprises topically administering to a subject in need thereof a nano formulation comprising a hormone or non-hormone according to the invention having a known therapeutic indication, such as anti-wrinkle and anti-aging properties.

### BACKGROUND OF THE INVENTION

### I. Background Regarding Nanoemulsions

Nanoemulsions as drug delivery systems are known. See e.g., US 2007-0264349 A1 for “Nano-structured compositions and methods of making and using the same”; and US 2008-0139513 A1 for “Transdermal deliver of active agents.” One of the challenges in developing a nanoemulsion formulation for drug delivery is stability of a formulation, both in terms of particle size stability and chemical stability. Emulsion formulations can grow in particle size over time, thus eliminating any benefits in formulation the emulsion as a nanoemulsion. Additionally, over time emulsion formulations can develop chemical degradation of the active agent to be delivered as well as chemical degradation of one or more emulsion components. Thus, there is a need in the art for nanoemulsion formulations having improved stability characteristics.

### II. Background Regarding Topical Skin Therapies

As a person ages, skin gets thinner, drier and less elastic. And it becomes less able to protect itself from damage. As a result, wrinkles, lines and creases form in the skin. Some wrinkles can become deep crevices or furrows and may be especially noticeable around the eyes, mouth and neck. McCullough et al., “Prevention and treatment of skin aging,” Annals of the New York Academy of Sciences, 1067:323 (2006). Other signs of aging skin include, but are not limited to, liver spots or age spots (solar lentigines), uneven skin tone, sun-damaged skin, and acne or chickenpox scars.

Wrinkles are caused by a combination of factors, such as age, exposure to ultraviolet (UV) light, smoking, and repeated facial expressions. Decreased production of natural oils makes skin drier and appear more wrinkled. Fat in the deeper layers of skin, which gives the skin a plump appearance, starts to lessen. This causes loose, saggy skin and more pronounced lines and crevices. See Helfrich et al., “Overview of skin aging and photoaging,” Dermatology Nursing, 20:177 (2008); Freiman et al., “Cutaneous effects of smoking,” Journal of Cutaneous Medicine and Surgery, 8:415 (2004); Just et al., “Effect of smoking on skin elastic fibres: Morphometric and immunohistochemical analysis,” British Journal of Dermatology, 156:85 (2007).

Current anti-aging treatments for skin include medications and surgical procedures and other techniques. However, all of the known treatments have disadvantages and undesirable side effects.

A. Medications and Cosmetics

Commercially available medications used to treat wrinkles and signs of skin aging include topical retinoids and non-prescription wrinkle creams. Topical retinoids are derived from vitamin A, are applied to the skin, and may be able to reduce fine wrinkles, splotchy pigmentation and skin roughness. Retinoids must be used with a skin-care program that includes sunscreen and protective clothing because the medication can make skin burn more easily. Retinoids may also cause redness, dryness, itching, and a burning or tingling sensation. Tretinoin (Renova®, Retin-A®) and tazarotene (Avage®, Tazorac®) are examples of topical retinoids. See Renova (tretinoin cream), U.S. Food and Drug Administration. http://fda.gov/ohrms/dockets/ac/02/slides/3904S1_{—}01_Bergfeld/sld024.htm (accessed Feb. 22, 2012); Kafi et al., “Improvement of naturally aged skin with vitamin A (retinol),” Archives of Dermatology, 143:606 (2007).

The effectiveness of non-prescription anti-wrinkle creams depends in part on the active ingredient or ingredients. Retinol, alpha hydroxy acids, kinetin, coenzyme Q10, copper peptides, and antioxidants may result in slight to modest improvements in wrinkles and signs of aging skin. Baumann L., “Cosmetics and skin care in dermatology,” In: Wolff et al., Fitzpatrick's Dermatology in General Medicine, 7th ed. (New York, N.Y.: The McGraw-Hill Companies; 2008). However, nonprescription wrinkle creams contain lower concentrations of active ingredients (such as alpha hydroxy acids) and other structurally different actives (such as retinoids) than do prescription creams. Therefore the claims and efficacy, if any, are limited and usually short-lived.

B. Surgical Procedures and Other Techniques

Surgical procedures and other techniques used to treat wrinkles and signs of aging skin include a variety of skin-resurfacing techniques, injectables, fillers and surgical procedures to smooth out wrinkles and treat signs of aging skin. Each works a little differently and has its own set of potential results and side effects. Examples include dermabrasion, microdermabrasion, laser, chemical peel, Botulinum toxin type A (Botox®), soft tissue fillers, face lift, and other surgical techniques.

Side effects and complications of Botox® injections include pain and bruising at the injection site, redness, headache, flu-like symptoms, nausea, temporary facial weakness or drooping, and spread of the toxin beyond the treatment area, which can cause botulism-like signs and symptoms (trouble swallowing, muscle weakness, slurred speech and breathing problems). Soft tissue fillers, which include fat, collagen and hyaluronic acid (Restylane®, Juvederm®), can be injected into deeper wrinkles on the face. They plump and smooth out wrinkles and furrows and give the skin more volume. Side effects include temporary swelling, redness and bruising in the treated area. The procedure may need to be repeated every few months.

Examples of documents describing compositions for treating wrinkles or skin aging include U.S. Pat. No. 6,896,889 for “Immediate effect anti-wrinkle composition, based on an aqueous dispersion, of at least one mineral filler,” directed to a composition comprising colloid particles of a mineral filler. U.S. Pat. No. 6,808,715 for “Wrinkle Cream,” is directed to an emulsion comprising water, hydrophilic particles, and hydrophobic particles, wherein the hydrophilic and hydrophobic particles form shells encapsulating a gas that is suspended in the water. U.S. Pat. No. 6,497,890 for “Anti-wrinkle preparation and method of reducing wrinkles in facial skin and neck,” is directed to a method for the prevention or minimization of wrinkles in the face and neck areas of a patient by topically applying finely divided safflower seeds or extract thereof in combination with a pharmaceutically acceptable carrier. U.S. Pat. No. 6,344,188 for “Wrinkle reducing cream,” is directed to a cream comprising water, caffeine, and glycerin. U.S. Pat. No. 5,360,824 for “Human skin cleansing and wrinkle-reducing cream,” is directed to a composition comprising water-soluble granules which can be an inorganic salt, such as a water-soluble vitamin and/or water-soluble vitamin-yielding salt, an oil and a petrolatum jelly. U.S. Pat. No. 4,777,041 for “Wrinkle treatment formulation,” is directed to compositions comprising a gelable hydrophilic polyurethane polymer base and a precipitated silica thickener gelling agent. U.S. Pat. No. 7,384,916 for “Methods and compositions for preventing and treating aging or photodamaged skin,” is directed to topical compositions comprising a peptide manganese complex. U.S. Pat. Nos. 7,354,610 and 7,214,395, both for “Pharmaceutical and cosmetic composition against skin aging,” is directed to compositions comprising phospholipid complexes of extracts of Vitis vinifera, and phospholipid complexes of standardized extract from Centella asiatica. U.S. Pat. No. 7,205,003 for “Method and topical formulation for treating skin conditions associated with aging,” is directed to topical compositions comprising a cosmeceutically active base, which is either an inorganic base, such as an inorganic hydroxide, an inorganic oxide, or a metal salt of a weak acid, or an organic base, such as a nitrogenous base.

### III. Background Regarding Hormone Drugs

The role of estrogen in the improvement of skin dates back to the 40s with the introduction of hormone replacement therapy for post-menopausal women. See Albright et al., “Postmenopausal osteoporosis: its clinical features,” JAMA, 116: 2465-74 (1941).

Estrogen receptors: There are two primary estrogen receptors (ER): (1) ER-α, which is predominantly found in reproductive target organs as a nuclear receptor (Lubahn et al., “Alteration of reproductive function but not prenatal sexual development after insertional disruption of the mouse estrogen receptor gene,” Proc. Natl. Acad. Sci. USA, 90(23):11162-6 (1993)); and (2) ER-β, which is predominantly found in reproductive organs, lungs and hypothalamus (Couse et al., “Tissue distribution and quantitative analysis of estrogen receptor-alpha (ERalpha) and estrogen receptor-beta (ERbeta) messenger ribonucleic acid in the wild-type and Ralphaknockoutmouse,” Endocrinology, 138 (11): 4613-21 (November 1997). Both types of receptors are found in the skin (Thornton et al., “Oestrogen receptor beta is the predominant oestrogen receptor in human scalp skin,” Exp Dermatol., 12(2):181-90 (2003)).

Women undergoing menopause have been found to have decreased numbers of ER; however, with hormone replacement therapy (HRT), women were found to have ER levels comparable to those of pre-menopausal women (Punnonen et al., “Demonstration of estrogen receptors in the skin,” J. Endocrinol. Invest., 3(3): 217-21 (1980)).

Estrogen and Collagen: In recent studies, topical estradiol treatment resulted in a 38% increase in hydroxyproline, a major constituent amino acid in collagen (Varila et al., “The effect of topical oestradiol on skin collagen of postmenopausal women,” Br. J. Obstet. Gynaecol., 102(12): 985-9 (1995)). Both human and animal studies have shown that estrogen modulates expression of collagen (via proliferation of dermal fibroblasts), and contributes to matrix metaloprotease down regulation which results in less collagen loss (Stevenson et al., “Effect of estrogens on skin aging and the potential role of SERMs,” Clin. Interv. Aging, 2(3): 283-97 (2007)). Clinical studies corroborate this finding where 18 postmenopausal women on 24 weeks of HRT were found to have 123% increase in dermal fibroblasts (Moraes et al., “The effects of topical isoflavones on postmenopausal skin: double-blind and randomized clinical trial of efficacy,” Eur. J. Obstet. Gynecol. Reprod. Biol., 146(2): 188-92 (2009)). Estrogen was also found to increase expression of glycosaminoglycans and proteoglycans, both of which are important constituents of the extracellular matrix. Furthermore, a large population based cohort study concluded in 1997 that topical estrogen use significantly prevented skin dryness (Dunn et al., “Does estrogen prevent skin aging? Results from the First National Health and Nutrition Examination Survey (NHANES I),” Arch. Dermatol., 133(3):339-42 (1997)).

Wrinkling characterized by loss of elasticity and ECM degradation: Histological studies show increased collagen degradation, and decreased numbers of sulfated glycosaminoglycans with the use of estrogen. In particular, women undergoing ERT were found to have improvements in elastic fibers (Varila et. al., Br. J. Obstet. Gynaecol., 102(12): 985-9 (1995)), and local treatment with estrogen to abdominal skin resulted in both increased numbers and thickness of elastic fibers, along with improved orientation of fibers (Punnonen et. al., J. Endocrinol. Invest., 3(3): 217-21 (1980)). Studies showed that estrogen deficient rats had delayed wound healing as seen by increased re-epithelialization time, wound width and collagen deposition. Similar studies used the same metrics found women with chronic estrogen deprivation showed the same changes (Ashcroft et al., “Estrogen accelerates cutaneous wound healing associated with an increase in TGF-beta1 levels,” Nat. Med., 3(11): 1209-15 (1997)). Moreover, topical estrogen seemed to reverse these deficits in wound healing (Ashcroft et al.).

While estrogen has been shown to have positive effects on skin aging, there are significant problems associated with the use of systemically absorbed estrogen. In particular, systemically absorbed estrogen is associated with cerebral vascular accidents (Anderson et al., “Effects of conjugated equine estrogen in postmenopausal Women with hysterectomy: the Women's Health Initiative randomized controlled trial,” JAMA, 291(14): 1701-12 (2004)). In addition, systemic administration of estradiol is also associated with breast and endometrial cancer (Prentice et al., “Benefits and risks of postmenopausal hormone therapy when it is initiated soon after menopause,” Am. J. Epidemiol., 170(1): 12-23 (2009)). In fact, Estrasorb®, which is the trade name for an estradiol topical gel and emulsion, has an FDA warning stating that: “Estradiol increases the risk that you will develop endometrial cancer (cancer of the lining of the uterus [womb]). The longer you use estradiol, the greater the risk that you will develop endometrial cancer.” Furthermore, the FDA warning for Estrasorb® also states that: “In a large study, women who took estrogens (a group of medications that includes estradiol) by mouth with progestins had a higher risk of heart attacks, strokes, blood clots in the lungs or legs, breast cancer, and dementia (loss of ability to think, learn, and understand). Women who use topical estradiol alone or with progestins may also have a higher risk of developing these conditions.” http://www.ncbi.nlm.nih.gov/pubmedhealth/PMH0000324/#.

Systemic absorption of estrogen is also associated with venous thromboembolism (Brandenberger et al., “Tissue distribution of estrogen receptors alpha (ER-alpha) and beta (ER-beta) mRNA in the midgestational human fetus,” J. Clin. Endocrinol. Metab., 82(10): 3509-12 (1997).

Thus, while it is known that a hormone such as estrogen when applied topically can have positive anti-aging effects, this is off-set by the significant side effects associated with systemic absorption of estrogen. There remains a need for more effective and otherwise improved methods for treating dermatological conditions related to aging skin, such as fine lines and wrinkles, and skin imperfections, such as scars. Additionally, there is a need in the art for nanoemulsion formulations useful as drug delivery vehicles, and having improved stability. The present invention addresses these needs and provides further related advantages.

### SUMMARY OF THE INVENTION

The present invention also provides nano hormonal formulations and methods of using the same for treating, preventing, and/or minimizing wrinkles, signs of aging skin, and/or skin imperfections, including but not limited to wrinkles, age spots, sun damage (particularly UV radiation-induced oxidative stress/photodamage), blemishes, hyperpigmented skin, age spots, increased skin thickness, loss of skin elasticity and collagen content, dry skin, lentigines, and melasmas. In contrast to prior art topical hormone formulations, such as Estrasorb®, the present invention is directed to topical hormonal nano formulations, such as a topical estrogen emulsion composition, which exhibits minimal or no systemic absorption. The hormonal nano emulsion compositions of the invention allow for targeted delivery to the area of skin to be treated.

The present invention provides hormonal nanoemulsion formulations having dramatically improved stability and shelf-life as compared to prior art hormonal nanoemulsion formulations.

Two specific formulation modifications were discovered to result in improved stability of a nano formulation according to the invention were discovered. The first relates to using super refined soybean oil along with the process of high pressure homogenization which allows for the production of a stable formulation. The second improvement relates to the use of a medium chain triglyceride oil, which also results in surprising and unexpected improvements in stability of the resultant composition. In another embodiment, the nano hormonal formulation of the invention can comprise a combination of an MCT oil and a super refined soybean oil.

In one embodiment of the invention, encompassed is a nano formulation according to the invention comprising estradiol and super refined soybean oil useful for the treatment of hot flush in postmenopausal women.

The compositions of the invention comprise at least one hormone, at least one surfactant, an aqueous phase, and squalene (or squalane), soybean oil (including super refined soybean oil), and/or medium chain triglyceride. Dilutions of the hormone emulsion compositions of the invention can also be used in the methods described herein. Surprisingly, it was discovered that the addition of squalene (or squalane), soybean oil (including super refined soybean oil), and/or medium chain triglyceride to an emulsion comprising a hormone appears to inhibit or block systemic absorption of the hormone present in the topically applied emulsion composition. This is significant as systemic absorption of hormones such as estradiol are associated with significant side effects. Examples of hormones that can be utilized in the claimed invention include, but are not limited to, any estrogen such as estradiol, genestein, testosterone, etc. The hormone can be synthetic or naturally occurring.

At least one surfactant can be employed in the compositions of the invention, for example, a pharmaceutically acceptable ionic surfactant, a pharmaceutically acceptable non-ionic surfactant, a pharmaceutically acceptable anionic surfactant, a pharmaceutically acceptable cationic surfactant, or a pharmaceutically acceptable zwitterionic surfactant.

In another embodiment, at least one antioxidant such as sodium metabisulfite or vitamin E can be utilized in the formulations of the invention. The antioxidant can be employed to maximize shelf life of the formulation.

In another embodiment, at least one microbial preservative such as methyl paraben, ethyl paraben, propyl paraben, or sorbic acid, can be utilized in the formulations of the invention. The microbial preservative can be employed to prevent microbial growth during the shelf life of the formulation.

In another embodiment, at least one viscosity promoting agent such as hydroxypropylmethylcellulose, methocel or ethocel can be utilized in the formulations of the invention. The viscosity promoting agent can be employed to optimize the formulation for topical administration.

Preferably the nano hormonal formulation of the invention comprise emulsion droplets with an average diameter of less than about 10 microns and up to about 1 nm.

In yet another embodiment of the invention, the nano hormonal formulation is thermostable.

Preferably, the hormonal nano formulations are in the form of any pharmaceutically acceptable dosage form, including but not limited to, ointments, creams, emulsions, lotions, gels, liquids, bioadhesive gels, dermal patch, sprays, shampoos, aerosols, pastes, foams, sunscreens, capsules, microcapsules, or in the form of an article or carrier, such as a bandage, insert, pessary, powder, talc or other solid, shampoo, cleanser, and agents that favor penetration within the epidermis, the dermis and keratin layers. Preferably, the hormonal nano formulations are in a dosage form suitable for topical administration. The hormonal nano formulation is capable of effectively treating and/or preventing signs of aging associated with the skin, such as fine to moderate wrinkles, uneven skin tone, and other dermatological signs of aging skin noted above, without being systemically absorbed and without significantly irritating the skin.

In a further embodiment, the hormonal nano formulation of the invention further comprises one or more non-hormone active agents. The presence of such an active agent is not required, and substantial and unexpected anti-aging and/or anti-wrinkle properties are observed without the presence of such a non-hormone active agent. Thus, the presence of such a non-hormone active agent is to merely enhance the anti-aging and/or anti-wrinkle properties of the hormonal nano formulation of the invention. In one embodiment, a non-hormone active agent incorporated into a hormone emulsion composition of the invention is a compound that provides a benefit to the skin and/or provides desirable properties to a composition formulated as a cosmetic or medicinal preparation. The non-hormone active agent can be a drug substance or a non-drug substance. Examples of non-hormone active agents that can be utilized in the compositions of the invention include, but are not limited to, skin lightening agents, tanning agents, skin conditioning agents, skin protectants, emollients and humectants, antiviral agents, NSAIDS, antifungal agents, topical steroids, and topical anesthetic agents.

In one embodiment, the nano hormonal formulation of the invention additionally comprises at least one ant-infective agent to promote rapid wound healing, particularly among diabetic patients. In another embodiment of the invention, the nano hormonal formulation of the invention additionally comprises at least one topical steroid to minimize or decrease the incidence of itching, rash and dermatitis. In yet another embodiment, the nano hormonal formulation of the invention additionally comprises at least one topical antifungal agent to relieve the complications of eczema, psoriasis and other dermatological maladies. In another embodiment of the invention, the nano hormonal formulation of the invention additionally comprises at least one topical NSAID to counter local pain, inflammation and discomfort. In yet another embodiment of the invention, the nano hormonal formulation of the invention additionally comprises at least one topical anesthetic agent to ease the pain and discomfort. In another embodiment, the nano hormonal formulation of the invention additionally comprises at least one topical antiviral agent to decrease the complication of oral and/or genital herpes-related symptoms.

Additional examples of non-hormone active agents, such as an active drug substance or an active cosmetic substance, that can be incorporated into a hormonal nano formulation of the invention include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac®), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids) or any mixture thereof.

The method of the invention comprises topically applying a hormonal formulation according to the invention, which is a non-invasive administration technique. Examples of signs of aging skin and/or skin imperfections which can be treated, prevented, and/or minimized with the methods of the invention include, but are not limited to, (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content (laxity and firmness), (10) melasmas (a typical pigmentation or hyper-pigmentation of the skin), (11) skin clarity and/or radiance, (12) skin smoothness and/or softness, (13) pore size (larger pore can make an individual appear older), (14) increase hydration, (15) increase skin smoothness, (16) increase skin tightness, (17) oral and/or genital herpes lesions, (18) skin wounds, (19) skin infections, and any combination thereof. The hormonal formulations of the invention can also be used to treat scars, such as acne and chickenpox scars. Collectively the signs of aging skin, skin imperfections and scars are referred to as “dermatological conditions.”

In an exemplary embodiment, a method of the invention for treating, reducing and/or minimizing the dermatological conditions described above (e.g., wrinkling, signs of aging skin, and/or skin imperfections) in a region of skin comprises applying a hormonal nano formulation according to the invention to the region of skin. The hormonal nano formulation can be applied to any skin region of a subject. In one embodiment, the hormonal nano formulation is applied to the facial tissue of a subject. In another embodiment, the hormonal nano formulation is applied to the neck tissue of a subject. In another embodiment the hormonal nano formulation is applied to the hand tissue of a subject. It has been surprisingly found that the hormonal nano formulations of the invention can be used to substantially treat, reduce, minimize, and/or diminish the dermatological conditions described above.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 shows the skin of an anesthetized male mouse prepped for testing with compositions according to the invention.

FIG. 2 shows the skin of a male anesthetized mouse following topical application of an estrogen formulation

FIG. 3 shows a pictorial of how tissues samples were obtained using a Metzenbaum curved scissors.

FIG. 4 shows the effect of treatment of copper/cobalt beats (HemogloBind®) to obtain hemoglobin-free plasma.

FIG. 5 shows a pictorial of a Cayment Chemical estradiol ELISA kit used to analyze plasma samples to determine estradiol amounts.

FIG. 6A shows a cross section of untreated mouse skin with wrinkles at the epidermis evident, and FIG. 6B shows a cross section of mouse skin tissue treated with diluted Estrasorb® (0.0025% w/w estradiol), with no wrinkles evident and hair follicle stem cells apparent.

FIG. 7A shows the results of picosirius red staining of an untreated mouse tissue sample, while FIG. 7B shows the results of picosirius red straining of a mouse tissue sample treated with Estrasorb® (0.0025% w/w estradiol), where tightly packed and better aligned collagen molecules are clearly evident. Picosirius red staining evaluates the presence and strength of collagen fibers.

FIG. 8A shows a cross section of untreated mouse skin having a thin epidermis, and FIG. 8B shows a cross section of mouse skin tissue treated with Estrasorb® (0.0025% w/w estradiol), with a thicker epidermis and hair follicle stem cells apparent.

FIG. 9A shows the results of picosirius red staining of an untreated mouse tissue sample, while FIG. 9B shows the results of picosirius red straining of a mouse tissue sample treated with a squalene low dose estradiol (0.005% w/w estradiol) nano formulation, where tightly packed and better aligned collagen molecules are clearly evident.

FIG. 10A shows the results of picosirius red staining of an untreated mouse tissue sample, while FIG. 10B shows the results of picosirius red straining of a mouse tissue sample treated with squalene high dose estradiol (0.25% w/w estradiol) nano formulation, where tightly packed and better aligned collagen molecules are clearly evident.

FIG. 11A shows the results of picosirius red staining of an untreated mouse tissue sample, while FIG. 11B shows the results of picosirius red straining of a mouse tissue sample treated with squalene placebo formulation, where no difference in the collagen fibers is apparent.

FIG. 12 depicts the results of an analysis of serum estradiol levels in mice topically treated with Estrasorb®, a squalene high dose estradiol nano formulation, a squalene low dose estradiol nano formulation, a squalene placebo, a Medium Chain Triglyceride (MCT) mid dose estradiol (0.025% w/w estradiol) nano formulation, and a MCT-placebo formulation.

FIG. 13 depicts a process and flow chart of manufacturing an estradiol nano formulation in soybean oil and super refined soybean oil.

FIG. 14 depicts a process and flow chart of manufacturing an estradiol nano formulation in medium chain triglyceride oil [MCT oil].

### DETAILED DESCRIPTION OF THE PREFERRED EMBODIMENTS

### I. Overview of the Invention

The present invention provides nanoemulsion formulations having dramatically improved stability and shelf life as compared to prior art nanoemulsion formulations. The nanoemulsion formulations can be used for drug delivery of a hormone or non-hormonal active agent.

The present invention also provides hormonal nano emulsion compositions and methods of using the same for treating, preventing, and/or minimizing wrinkles, signs of aging skin, and/or skin imperfections.

The compositions of the invention comprise at least active agent such as a hormone or non-hormone, a surfactant such as a poloxamer, an aqueous phase, and squalene, soybean oil (including super refined soybean oil), and/or or medium chain triglyceride. Surprisingly, it was discovered that the addition of squalene (or squalane), soybean oil (including super refined soybean oil), and/or medium chain triglyceride (MCT) to a nano formulation of a hormone or non-hormone appears to inhibit or block systemic absorption of the active agent when the formulation is topically applied to skin tissue.

Moreover, it was also surprisingly discovered that the presence of an oil such as MCT results in dramatically improved stability as compared to other oils, such as castor oil or mineral oil.

The hormonal nano formulations can be in the form of any pharmaceutically acceptable dosage form which can be topically applied, including but not limited to, liquids, ointments, creams, oils, emulsions, lotions, gels, liquids, bioadhesive gels, dermal patches, sprays, shampoos, aerosols, pastes, foams, or in the form of an article or carrier, such as a bandage, insert, powder, talc or other solid. The hormonal nano formulations are capable of effectively treating, preventing, and/or minimizing the dermatological conditions described herein, without being systemically absorbed and without significantly irritating the skin.

A. Inhibition of Systemic Absorption

Surprisingly, it was discovered that the addition of squalene (or squalane), soybean oil (including super refined soybean oil), and/or medium chain triglyceride (MCT) to a hormonal nano formulation effectively eliminates or minimizes systemic absorption of the component hormone when the formulation is topically applied to skin tissue. Even more surprising was the discovery that although systemic delivery of the component hormone is minimized or eliminated, the local effects of the hormone are retained. The topical hormonal nano formulations of the invention effectively treat epidermic conditions, such as wrinkles, without the risks associated with systemic absorption into the blood stream, thereby, minimizing or negating any systermic side effects associated with the hormone.

In particular, the results of Example 2 demonstrate that treatment with a topical squalene estrogen nano formulation according to the invention does not result in significant systemic absorption of the component hormone, e.g., estradiol. Specifically, the data shows that there appears to be little if any systemic absorption of estradiol via topical application of a squalene estradiol nano formulation according to the invention, as determined by plasma estradiol concentration determined by ELISA assay. In contrast, topical application of Estrasorb® results in dramatic increases in serum estradiol. Thus, with a topical hormonal formulation lacking squalene (or squalane) or MCT, the systemic side effects associated with estradiol are manifested and remain in full force.

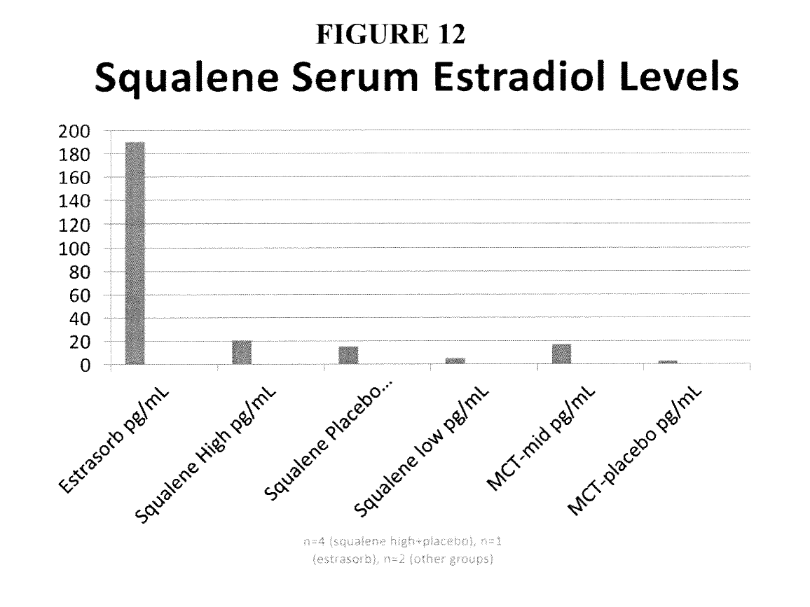

As detailed in Example 2 below, Estrasorb®, which is designed to deliver estradiol systemically, exhibited very high levels of serum estradiol. See FIG. 12. However, in surprising contrast, a topically applied squalene low dose estradiol nano formulation according to the invention exhibited a lower serum estradiol level than the squalane placebo formulation! See FIG. 12. (This is possible as estradiol is naturally present in the blood stream even in male mice.) Moreover, all of the formulations tested according to the invention, namely (1) a squalene high dose estradiol nano formulation, (2) a squalene low dose estradiol nano formulation, and (3) a MCT-mid dose estradiol nano formulation, exhibited minimal serum levels of estradiol, which were about the same or less than the serum estradiol levels measured for the squalene placebo composition. See FIG. 12.

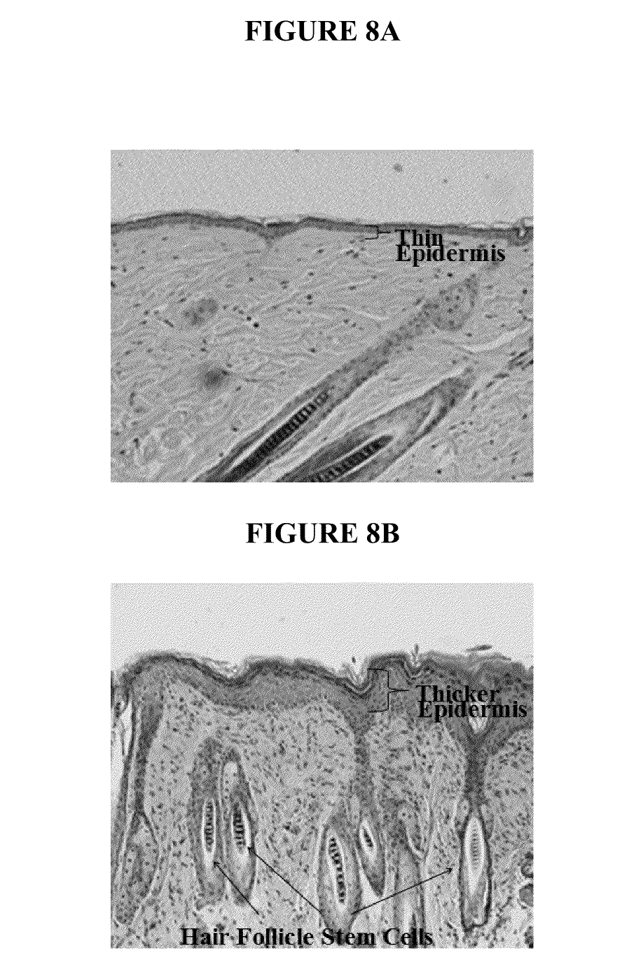

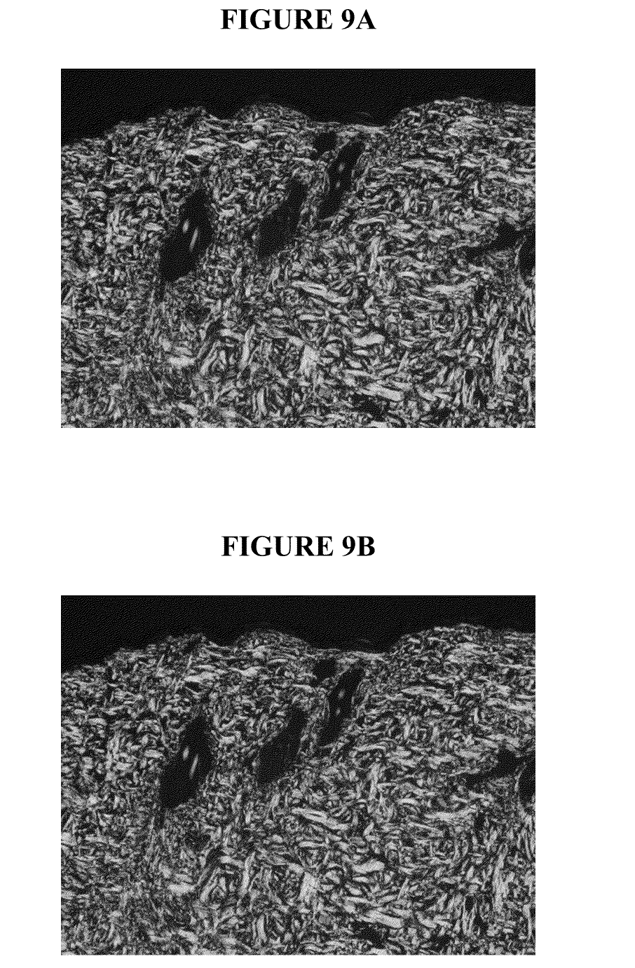

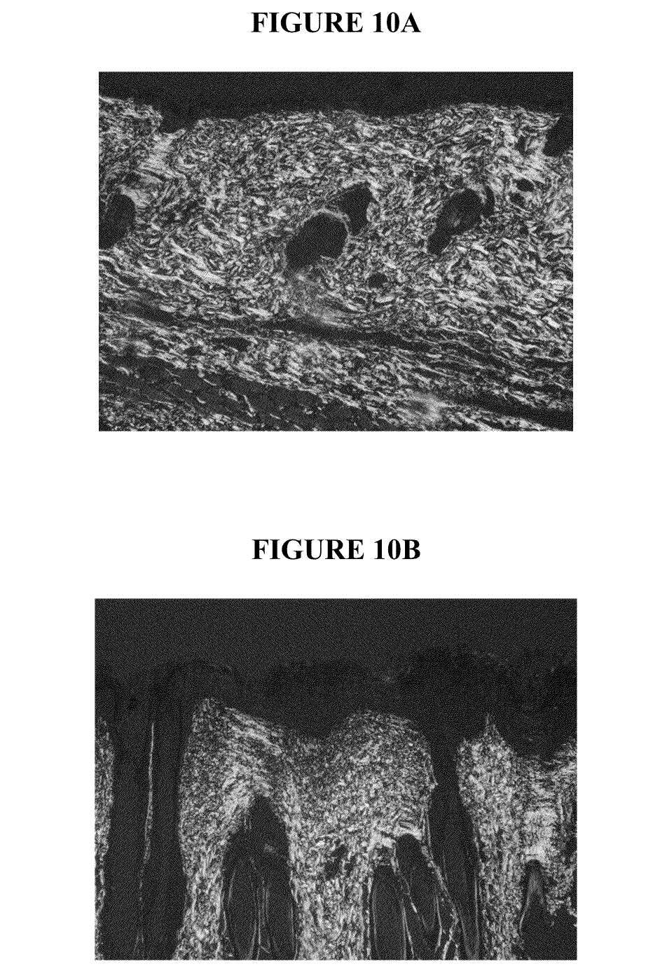

These results are exceptional as while the hormonal nano formulations according to the invention exhibited minimal or no systemic absorption of estradiol, they also exhibited significant localized effects in terms of thickening the epidermis (FIG. 8B; 0.25% estradiol squalene nano formulation), stimulating the production of hair follicle stem cells (FIG. 8B; 0.25% estradiol squalene nano formulation), and stimulating the production of collagen molecules (FIG. 9B; 0.025% estradiol squalene nano formulation; and FIG. 10B; 0.25% estradiol squalene nano formulation).

The systemic absorption of a hormonal formulation lacking a medium chain triglyceride or squalene, such as Estrasorb®, was evident by the non-localized effects of the topically applied formulation. Specifically, topical application of Estrasorb® resulted in thickening of skin which was not limited to the site of application. In contrast, application of a squalene estradiol nano formulation according to the invention resulted in only localized effects, thereby additionally substantiating the lack of systemic absorption of the hormone when topically applied in a nano formulation according to the invention.

Another example of a hormone that can be utilized in the nano formulations of the invention is testosterone. Formulating the male hormone testosterone using squalene, soybean oil (including super refined soybean oil), and/or MCT oil results in avoiding or minimizing systemic absorption of the drug into the blood stream following topical application. Thus, all of the localized benefits associated with topical application of testosterone, such as in the case of aging, skin rejuvenation, moisturizing, etc. particularly among male subjects, are retained with the formulation of the invention without any side effects associated with systemic absorption of testosterone. In contrast, the current commercial formulation of testosterone, Androgel® 1% topical formulation of testosterone, which is primarily formulated in ethanol and other additives, is systemically absorbed approximately to the extent of 10%. The medical indication for Androgel® 1% is related to transdermal delivery of testosterone into the blood stream for the treatment of male hypogonadism; e.g., the current commercial formulation is designed for systemic delivery of the active agent. Additionally, there is evidence of off-label use of Androgel® 1% in treating male erecticle dysfunction [ED] cases.

The data described herein demonstrate that hormones topically applied to the skin in a nano formulation according to the invention result in increased dermal or epidermis thickness, reduced wrinkles, increased stem cell hair follicles, and increased, more densely packed, and more organized collagen fibers. However, while the Estrasorb® application led to thickening of skin at the site of application and elsewhere, the effects of the squalene estradiol nano formulation were localized to the site of application. This result was highly unexpected, unique, and not obvious to one skilled in the art.

Thus, the hormonal nano formulations of the invention result in delivery of a hormone which remains in the epidermal layer and does not traverse into the dermal layer. Entry of the hormone into the blood stream is blocked via the formulation of the invention, with the net result being avoidance of systemic side effects caused by the hormone drugs either via conventional transdermal or oral delivery systems.

B. Improved Formulation Stability

Two specific formulation modifications resulting in improved stability of a nano formulation according to the invention were discovered. The first relates to using super refined soybean oil (e.g., Cropure™ Soybean, Super Refined™ Soybean, Super Refined™ Soybean EP NP, Super Refined™ Soybean USP, Super Refined™ Soybean USP NP, Super Refined™ Soybean USP JP NP) along with the process of high pressure homogenization which allows for the production of a stable formulation. See also http://www.destecs.com/Soybeanoil.html, which provides an exemplary profile of a super refined soybean oil. Preferably super refined soybean oil has less than about 10% impurities. The second improvement relates to the use of a medium chain triglyceride oil, which also results in surprising and unexpected improvements in stability of the resultant composition. In another embodiment, the nano hormonal formulation of the invention can comprise a combination of an MCT oil and a super refined soybean oil.

It was surprisingly discovered that the use of a super refined soybean oil having less polar impurities than conventional soybean oil results in a dramatic improvement in stability of a hormonal formulation according to the invention. In particular, the use of a super refined soybean oil, preferably combined with the process of high pressure homogenization to make the formulation, results in a commercial shelf life of at least 2 plus years. Regular soybean oil contains high levels of polar impurities which can result in an unstable formulation with respect to: (1) physical phase separation of the emulsion, (2) growth of the particle size of the droplets forming the emulsion, as well as (3) chemical degradation of the active agent and/or components of the emulsion formulation within 2 months of accelerated storage at 40° C./75% relative humidity storage. An exemplary method of making a nano formulation comprising super refined soybean oil suitable as a delivery vehicle for a hormone is described in Example 3.

A second method of improving the stability of a hormonal nano formulation of the invention is by utilizing a medium chain triglyceride (MCT) in the formulation. An example of an MCT is Miglyol® 812, which comprises 55% triglycerides of C₈ and 45% triglycerides of C₁₀ fatty acids. An exemplary method of making a nano formulation comprising an MCT suitable as a delivery vehicle for a hormone is described in Example 4.

Medium-chain triglycerides (MCTs) are medium-chain (6 to 12 carbons) fatty acid esters of glycerol. Coconut oil is composed of approximately 66% medium-chain triglycerides. Other rich sources of MCTs include palm kernel oil and camphor tree drupes. The fatty acids found in MCTs are called medium-chain fatty acids. The names of the medium-chain fatty acids (and the corresponding number of carbons) found in MCTs are caproic acid (C₆), caprylic acid (C₈), capric acid (C₁₀) and lauric acid (C₁₂). Like all triglycerides (fats and oils), MCTs are composed of a glycerol backbone and three fatty acids, hence the name triglyceride; in the case of MCTs, 2 or 3 of the fatty acid chains attached to glycerol are medium chain in length. The approximate ratios of these fatty acids in commercial MCT products derived from coconut oil are 2(C₆):55(C₈):42(C₁₀):1(C₁₂).

C. Therapeutic Effectiveness of the Compositions

Examples of signs of aging skin and/or skin imperfections which can be treated, prevented, and/or minimized with the methods of the invention include, but are not limited to, (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content, (10) melasmas (atypical pigmentation or hyper-pigmentation of the skin), (11) skin clarity and/or radiance, (12) skin smoothness and/or softness, (13) pore size (larger pore can make an individual appear older), (14) increase hydration, (15) increase skin smoothness, (16) increase skin tightness, (17) oral and/or genital herpes lesions, (18) skin wounds, (19) skin infections, and any combination thereof. The compositions of the invention can also be used to treat scars, such as acne and chickenpox scars. Collectively the signs of aging skin, skin imperfections and scars are referred to as “dermatological conditions.”

In an exemplary embodiment, a method of the invention for treating, reducing and/or minimizing dermatological conditions in a region of skin comprises topically applying a hormonal nano formulation according to the invention to a region of skin. In one embodiment, the hormonal nano formulation is applied to the facial tissue of a subject. In another embodiment, the hormonal nano formulation is applied to the neck tissue of a subject. In yet another embodiment the hormonal nano formulation is applied to the hand tissue of a subject. It has been surprisingly found that the hormonal nano formulations of the invention can be used to substantially treat, minimize, and/or diminish the dermatological conditions described above.

D. Combination Therapy

In a further embodiment, the hormonal nano formulations of the invention can additionally comprise one or more non-hormone active agents. The presence of such a non-hormone active agent is not required, and substantial and unexpected anti-aging and/or anti-wrinkle properties are observed without the presence of such a non-hormone active agent. Thus, the presence of such non-hormone active agents is to merely enhance the anti-aging and anti-wrinkle properties of the hormonal nano formulations of the invention. In one embodiment, non-hormone active agents useful in the hormonal nano formulations of the invention are compounds that provide benefits to the skin and/or provide desirable properties to a composition formulated as a cosmetic or medicinal preparation. The non-hormone active agent useful in the hormonal nano formulations of the invention can be a drug substance or a non-drug substance. Examples of non-hormone, non-drug active agents include, but are not limited to, skin lightening agents, tanning agents, skin conditioning agents, skin protectants, emollients, humectants, antiinfectives, topical steroids, antifungals, nonsteroidal anti-inflammatory agents, anesthetic agents, and/or antiviral agents.

These non-hormonal therapeutic category of drugs, combined with a hormonal agent in a nano formulation according to the invention, are applicable for acute or short term usage in topical, local actions with complimentary benefits to maximize treatment outcome.

Examples of exemplary non-hormone active agents useful in the hormonal nano formulations of the invention, such as an active drug substance or an active cosmetic substance, include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac®), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids) or any mixture thereof.

Nano formulations comprising at least one hormone in combination with at least one antiinfective agent are useful for the rapid onset and enhanced treatment outcome of wound healing. This is particularly true in the case of diabetics where wounds can have difficulty healing properly due to side effects associated with diabetes. This is a significant improvement over prior art therapies, as flesh wounds in diabetics can require surgical intervention, skin grafting or loss of body parts. Examples of antiinfectives that can be utilized in a nano hormonal formulation of the invention include, but are not limited to, tobramycin, ciprofloxacin, nafcillin, amikacin, gentamicin, kanamycin, neomycin, netilmicin, tobramycin, paromomycin, spectinomycin, geldanamycin, herbimycin, rifaximin, loracarbef, ertapenem, doripenem, imipenem/cilastatin, meropenem, cefadroxil, cefazolin, cefalotin or cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, ticarcillin, amoxicillin/clavulanate, ampicillin/sulbactam, piperacillin/tazobactam, ticarcillin/clavulanate, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine (archaic), sulfacetamide, sulfadiazine, silver sulfadiazine, sulfamethizole, sulfamethoxazole, sulfanilimide (archaic), sulfasalazine, sulfisoxazole, trimethoprim-Sulfamethoxazole (Co-trimoxazole) (TMP-SMX), demeclocycline, doxycycline, minocycline, oxytetracycline, tetracycline, clofazimine, dapsone, capreomycin, cycloserine, ethambutol, ethionamide, isoniazid, pyrazinamide, rifampicin (Rifampin in US), rifabutin, rifapentine, streptomycin, arsphenamine, chloramphenicol, fosfomycin, fusidic acid, metronidazole, mupirocin, platensimycin, quinupristin/Dalfopristin, thiamphenicol, tigecycline, tinidazole, and trimethoprim.

Nano formulations comprising at least one hormone in combination with at least one topical steroid agent are useful to decrease the incidence of local rash and inflammation. Examples of topical steroid agents that can be utilized in a nano hormonal formulation of the invention include, but are not limited to, betamethasone, triamcinolone, hydrocortisone, prednisolone, clobetasol, halobetasol, diflorasone, fluocinonide, halcinonide, amcinonide, desoximetasone, mometasone, fluticasone, fluocinolone acetonide, hydrocortisone valerate, hydrocortisone butyrate, flurandrenolide, triamcinolone acetonide, mometasone furoate, fluticasone propionate, desonide, alclometasone dipropionate, and fluocinolone acetonide.

Nano formulations comprising at least one hormone in combination with at least one antifungal agent are useful to decrease local complications such as eczema, dermatitis, psoriasis etc. Examples of antifungal agents that can be utilized in a nano hormonal formulation of the invention include, but are not limited to, (1) azoles (imidazoles), (2) antimetabolites, (3) allylamines, (4) morpholine, (5) glucan synthesis inhibitors (chemical family: echinocandins), (6) polyenes, (7) benoxaborales, (8) other antifungal agents, and (9) new classes of antifungal agents. Examples of azoles include, but are not limited to, Bifonazole, Clotrimazole, Econazole, Miconazole, Tioconazole, Fluconazole, Itraconazole, Ketoconazole, Pramiconazole, Ravuconazole, Posaconazole, and Voriconazole. An example of an antimetabolite includes, but is not limited to, Flucytosine. Examples of allylamines include, but are not limited to, Terbinafine and Naftidine and amorolfine. Examples of glucan Synthesis Inhibitors include, but are not limited to, Caspofungin, Micafungin, and Anidulafungin. Examples of polyenes include, but are not limited to, Amphotericin B, Nystatin, and pimaricin. An example of a benoxaborale is AN2690. Other examples of antifungal/onychomycosis agents include, but are not limited to, griseofulvin and ciclopirox. Finally, examples of new classes of antifungal/onychomycosis agents include, but are not limited to, sodarin derivatives and nikkomycins.

Nano formulations comprising at least one hormone in combination with at least one antiviral agent are useful to counter herpes (HSV-1 and/or HSV-2) associated ailments, including oral and genetal herpes infections. Examples of antiviral agents that can be utilized in a nano hormonal formulation of the invention include, but are not limited to, nucleoside analogs (e.g., acyclovir (Zovirax®), famciclovir (Famvir®), and valaciclovir (Valtrex®)), amantadine (Symmetrel®), oseltamivir (Tamiflu®), rimantidine (Flumadine®), and zanamivir (Relenza®), Cidofovir (Vistide®), foscarnet (Foscavir®), ganciclovir (Cytovene®), ribavirin (Virazole®), penciclovir (Denavir®), buciclovir, acyclic guanosine derivatives, (E)-5-(2-bromovinyl)-2′-deoxyuridine and structurally related analogues thereof [i.e., the cytosine derivative (E)-5-(2-bromovinyl)-2′-deoxycytidine and the 4′-thio derivative (E)-5-(2-bromovinyl)-2′-deoxy-4′-thiouridine], Nucleoside/Nucleotide Analogues (e.g., Abacavir (Ziagen, ABC), Didanosine (Videx, ddI), Emtricitabine (Emtriva, FTC), Lamivudine (Epivir, 3TC), Stavudine (Zerit, d4T), Tenofovir (Viread, TDF), Zalcitabine (Hivid, ddC), and Zidovudine (Retrovir, AZT, ZDV)); Nonnucleoside Reverse Transcriptase Inhibitors (e.g., Delavirdine (Rescriptor, DLV), Efavirenz (Sustiva, Stocrin, EFV), Etravirine (Intelence, TMC 125), Nevirapine (Viramune, NVP)); Protease Inhibitors (Amprenavir (Agenerase, APV), Atazanavir (Reyataz, ATV), Darunavir (Prezista, DRV, TMC 114), Fosamprenavir (Lexiva, Telzir, FPV), Indinavir (Crixivan, IDV), Lopinavir/Ritonavir (Kaletra), Nelfinavir (Viracept, NFV), Ritonavir (Norvir, RTV), Saquinavir (Invirase, SQV), and Tipranavir (Aptivus, TPV)); Fusion Inhibitors (e.g., Enfuvirtide (Fuzeon, ENF, T-20)); Chemokine Coreceptor Antagonists (e.g., Maraviroc (Selzentry, Celsentri, MVC)); and Integrase Inhibitors (e.g., Raltegravir (Isentress, RAL)). Preferred antiviral agents for incorporation into a nanoemulsion include, but are not limited to, acyclovir (Zovirax®), penciclovir (Denavir®), famciclovir (Famvir®), and valaciclovir (Valtrex®).

Nano formulations comprising at least one hormone in combination with at least one a nonsteroidal anti-inflammatory (NSAID) agent are useful to decrease local pain and inflammation. Examples of NSAIDS that can be utilized in a nano hormonal formulation of the invention include, but are not limited to, Aspirin, Choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, and valdecoxib.

Nano formulations comprising at least one hormone in combination with at least one anesthetic agent are useful to numb an area of local discomfort and pain. Examples of anesthetic agents that can be utilized in a nano hormonal formulation of the invention include, but are not limited to, lidocaine, bupivacaine, mepivicaine, epinephrine, tetracaine, pramoxine, cetacaine, prilocalne, exactacain, pramosone, procaine (Novocain), benzocaine, etidocaine, benzyl alcohol, phenol, resorcinol and juniper tar.

These non hormonal therapeutic category of drugs combined with a hormonal agent are applicable for acute or short term usage in topical, local actions with complimentary benefits to maximize treatment outcome not disclosed in prior teachings.

### II. Definitions

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 20% of the particular term, or plus or minus 10% of the particular term.

The term “active agent” is used herein to refer to a chemical material or compound that induces a desired beneficial effect when administered topically or subcutaneously, and includes agents that are therapeutically and/or prophylactically effective as pharmaceuticals (“pharmacologically active agents”), as well as agents that are cosmeceutically effective (“cosmeceutically active agents”). Also included are derivatives and analogs of those compounds or classes of compounds specifically mentioned that also induce the desired effect. An “effective” amount of an active agent means a nontoxic but sufficient amount of an active agent to provide the desired beneficial effect. More specifically, a “therapeutically effective,” “prophylactically effective,” or “cosmeceutically effective” amount means a nontoxic but sufficient amount of a beneficial agent to provide the desired therapeutic, prophylactic, or cosmeceutical effect.

The term “aging-related skin condition” relates to any skin condition or disorder associated with, caused by, or affected by, intrinsic aging and/or extrinsic aging. Aging-related skin conditions that may be treated using the present methods and formulations include, but are not limited to, wrinkles, age spots, sun damage (particularly UV radiation-induced oxidative stress), blemishes, hyperpigmented skin, age spots, increased skin thickness, loss of skin elasticity and collagen content, dry skin, lentigines, melasmas, as well as scars.

The terms “buffer” or “buffering agents” refer to materials which when added to a solution, cause the solution to resist changes in pH.

“Carriers” or “vehicles” as used herein refer to carrier materials suitable for incorporation in a topically or subcutaneously applied composition. Carriers and vehicles useful herein include any such materials known in the art, which are nontoxic and do not interact with other components of the formulation in which it is contained in a deleterious manner.

By “cosmeceutically effective” is meant a nontoxic agent that has medicinal or drug-like properties which, when applied to the surface of skin, beneficially affects the biological functioning of that skin.

The terms “cosmeceutically active agent” and “cosmeceutically active base” are used interchangeably herein to refer to a cosmeceutically effective basic compound or composition of matter which, when topically administered to a human patient, is effective to treat one or more aging-related skin conditions as defined above. Also included are derivatives and analogs of those compounds or classes of compounds specifically mentioned that also induce the desired effect, i.e., treatment of an aging-related skin condition.

“Cosmeceutically acceptable,” such as in the recitation of a “cosmeceutically acceptable carrier,” or a “cosmeceutically acceptable derivative,” means a compound that is not biologically or otherwise undesirable, i.e., the compound may be incorporated into a cosmeceutical formulation of the invention and topically administered to a patient without causing any undesirable biological effects or interacting in a deleterious manner with any of the other components of the cosmeceutical formulation in which it is contained. The term “pharmaceutically acceptable” is used in an analogous manner, to refer to a compound or composition that may be incorporated into a pharmaceutical formulation herein (i.e., a cosmeceutical formulation containing one or more pharmacologically active agents) without causing undesirable biological effects or unwanted interaction with other components of the formulation.

The term “dilution” refers to dilution of the hormonal nano formulations of the present invention or those derived from the hormonal nano formulations of the present invention using, for example, an aqueous system comprised of physiologically balanced saline solution (PBS), such as phosphate buffered saline, or water, or other water soluble components, to the desired final concentration.

The term “emulsion,” as used herein, includes dispersions or droplets, as well as other lipid structures that can form as a result of hydrophobic forces that drive a polar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The droplets have an average diameter of less than about 10 microns.

The terms “pharmaceutically acceptable” or “pharmacologically acceptable,” as used herein, refer to compositions that do not substantially produce adverse allergic or immunological reactions when administered to a host (e.g., an animal or a human). Such formulations include any pharmaceutically acceptable dosage form. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The term “stable” when referring to a “stable hormonal nano formulation” means that the formulation retains its structure as an emulsion. A desired emulsion structure, for example, may be characterized by a desired size range, macroscopic observations of emulsion science (is there one or more layers visible, is there visible precipitate), pH, and a stable concentration of one or more of the components.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group. Other surfactant include non-ionic and zwitterionic surfactants.

As used herein, the term “topically” refers to application of the compositions of the present invention to the surface of the skin and tissues.

The terms “treating” and “treatment” as used herein refer to reduction in severity and/or elimination of skin related conditions resulting from intrinsic and/or extrinsic aging processes of the skin, or other trauma to the skin resulting in, e.g., a scar. The present method of “treating” a skin condition related to aging, as the term is used herein, refers to the prevention of aging-related skin conditions as well as the treatment of aging-related skin conditions in affected individuals.

### III. Compositions of the Invention

The hormonal nano formulations of the invention comprise (i) at least one hormone, (ii) at least one surfactant, (iii) an aqueous phase, and (iv) squalene (or squalane), soybean oil (including super refined soybean oil), and/or medium chain triglyceride, or a combination of squalene (or squalane), soybean oil (including super refined soybean oil), and/or medium chain triglyceride. Additionally, the hormonal nano formulation can optionally comprise one or more non-hormone active agents, as detailed infra, and/or other pharmaceutically acceptable excipients such as antioxidants and microbial preservatives. Surprisingly, it was discovered that the addition of squalene (or squalane), soybean oil (including super refined soybean oil), and/or medium chain triglyceride to a nano formulation comprising a hormone appears to inhibit or block systemic absorption of the hormone present in the topically applied formulation.

In one embodiment of the invention, the at least one hormone is present in an amount ranging from about 0.0001% to about 15.0% (wt. %), the at least one surfactant is present in an amount ranging from about 0.01% to about 50% (wt. %), the aqueous phase is present in an amount ranging from about 10% to about 90% (wt. %), and squalene (or squalane) is present in an amount ranging from about 10% to about 85% (wt. %), based on the total weight of the emulsion composition. In another embodiment of the invention, the at least one hormone is present in an amount ranging from about 0.001% to about 5.0% (wt. %), the at least one surfactant is present in an amount ranging from about 3% to about 10% (wt. %), the aqueous phase is present in an amount ranging from about 40% to about 60% (wt. %), and squalene (or squalane) is present in an amount ranging from about 40% to about 65% (wt. %), based on the total weight of the emulsion composition.

In one embodiment of the invention, the at least one hormone is present in an amount ranging from about 0.0001% to about 15.0% (wt. %), the at least one surfactant is present in an amount ranging from about 0.01% to about 50% (wt. %), the aqueous phase is present in an amount ranging from about 10% to about 90% (wt. %), and medium chain triglyceride is present in an amount ranging from about 10% to about 85% (wt. %), based on the total weight of the emulsion composition. In another embodiment of the invention, the at least one hormone is present in an amount ranging from about 0.001% to about 5.0% (wt. %), the at least one surfactant is present in an amount ranging from about 3.0% to about 10% (wt. %), the aqueous phase is present in an amount ranging from about 40% to about 60% (wt. %), and medium chain triglyceride is present in an amount ranging from about 40% to about 65% (wt. %), based on the total weight of the emulsion composition.

In one embodiment of the invention, the at least one hormone is present in an amount ranging from about 0.0001% to about 15.0% (wt. %), the at least one surfactant is present in an amount ranging from about 0.01% to about 50% (wt. %), the aqueous phase is present in an amount ranging from about 10% to about 90% (wt. %), and soybean oil (including super refined soybean oil) is present in an amount ranging from about 10% to about 85% (wt. %), based on the total weight of the emulsion composition. In another embodiment of the invention, the at least one hormone is present in an amount ranging from about 0.001% to about 5.0% (wt. %), the at least one surfactant is present in an amount ranging from about 3.0% to about 10% (wt. %), the aqueous phase is present in an amount ranging from about 40% to about 60% (wt. %), and soybean oil (including super refined soybean oil) is present in an amount ranging from about 40% to about 65% (wt. %), based on the total weight of the emulsion composition.

The hormonal nano formulations of the invention can be diluted to any desired hormone concentration, while maintaining stability of the emulsion formulation.

Several exemplary hormonal nano formulations according to the invention are shown below in Tables 1 and 2.

TABLE 1
Exemplary Hormonal Nano Formulation Comprising MCT

| | | Medium | |
|---|---|---|---|
| | High Estradiol | Estradiol | Low Estradiol |
| Ingredient | Concentration | Concentration | Concentration |
| Estradiol | 250 mg | 25 mg | 5 mg |
| Pluronic F 68 | 6 g | 6 g | 6 g |
| Medium Chain | 47 g | 47 g | 47 g |
| Trigyceride | | | |
| Purified Water | 46.75 g | 46.95 g | 46.995 g |

TABLE 2
Exemplary Hormonal Nano Formulation Comprising Squalene

| Ingredient | 3H | 3M | 3l |
|---|---|---|---|
| Estradiol | 250 mg | 25 mg | 5 mg |
| Pluronic F68 | 5 g | 5 g | 5 g |
| Squalene | 52 g | 52 g | 52 g |
| Purified Water | 42.75 g | 42,975 g | 42.995 g |

A. Components of the Emulsion Compositions of the Invention

1. Hormones

Any pharmaceutically acceptable hormone can be used in the compositions of the invention, including naturally occurring hormones as well as synthetic hormones, hormone analogs, and hormone derivatives.

The hormonal nano formulation is preferably stored at between about 2° C. and about 40° C. At room temperature (e.g., about 25° C.) the hormonal nano formulation can be stored for up to about 3 years without exhibiting any degradation or instability. This long stability is useful to support the shelf life of the formulation.

Examples of suitable hormones include but are not limited to estrogens and progestagens. Estrogens include but are not limited to estrone, estradiol, estriol, genestein, equilin, equilenin, chlorotrianisene, dienestrol, diethylstilbestrol, ethinylestradiol, fosfestrol, mestranol, polyestradiol phosphate, xenoestrogens, phytoestrogens, and mycoestrogens, and progestagens include but are not limited to progesterone, desogestrel, drospirenone, dydrogesterone, ethisterone, etonogestrel, ethynodiol diacetate, gestodene, gestonorone, levonorgestrel, lynestrenol, medroxyprogesterone, megestrol, norelgestromin, norethisterone, norethynodrel, norgestimate, norgestrel, norgestrienone, and tibolone. Other hormones useful in the compositions of the invention include but are not limited to Melatonin (N-acetyl-5-methoxytryptamin) and testesterone.

2. Surfactant

Any pharmaceutically acceptable surfactant may be utilized in the hormonal nano formulations of the invention, including pharmaceutically acceptable ionic, non-ionic, anionic, cationic, and zwitterionic compounds or surfactants. Exemplary surfactants that may be used in the hormonal nano formulations of the invention include, but are not limited to, non-phospholipid surfactants, such as the Tween family of surfactants (polyoxyethylene derivatives of sorbitan fatty acid esters; e.g., Tween 20, Tween 60, and Tween 80), nonphenol polyethylene glycol ethers, sorbitan esters (such as Span and Arlacel), glycerol esters (such as glycerin monostearate), polyethylene glycol esters (such as polyethylene glycol stearate), poloxamers or block polymers (such as Pluronics, e.g., Pluronic F68), acrylic polymers (such as Pemulen), ethoxylated fatty esters (such as Cremophor RH-40), ethoxylated alcohols (such as Brij), ethoxylated fatty acids, monoglycerides, silicon based surfactants, polysorbates, Tergitol NP-40 (Poly(oxy-1,2-ethanediyl), α-(4-nonylphenol)-.omega.-hydroxy, branched [molecular weight average 1980]), and Tergitol NP-70 (a mixed surfactant—AQ=70%).

In another embodiment, the surfactant is selected from the group consisting of, but not limited to, sorbitan esters, glycerol esters, polyethylene glycol esters, poloxamers, block polymers, acrylic polymers (such as Pemulen), ethoxylated fatty esters (such as Cremophor RH-40), ethoxylated alcohols (such as Brij), ethoxylated fatty acids (such as Tween), monoglycerides, silicon based surfactants, and polysorbates.

Preferably, the individual surfactant molecules are free of cross-linkages. The surfactant is also preferably soluble in water. One or more surfactants may be used in the compositions and methods of the invention. As used herein, the terms “stabilizer”, “surface stabilizer”, and “surfactant” are used interchangeably.

3. Squalene, Soybean Oil and/or Medium Chain Triglyceride

The hormonal nano formulations of the invention also comprise squalene (or squalane), soybean oil (including super refined soybean oil), or medium chain triglyceride; the compositions can also comprise a combination of these compounds.

Squalene is a natural organic compound originally obtained for commercial purposes primarily from shark liver oil, though plant sources (primarily vegetable oils) are used as well. All plants and animals produce squalene, including humans. Squalane is a saturated form of squalene in which the double bonds have been eliminated by hydrogenation. Both squalene and squalane can be used in the compositions of the invention. Both naturally occurring and synthetic forms of squalene and squalane can be used in the compositions of the invention, including derivatives of squalene or squalane. Synthetic and naturally occurring squalenes and squalanes are collectively referred to herein as “squalene.”

Squalene is made up of six (trans-1,4)-isoprene units linked as two farnesyl (head-to-tail) groups that are joined tail to tail in the center:

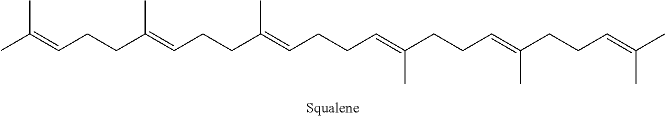 [SMILES: CC(C)=CCC/C(C)=C/CC/C(C)=C/CC/C=C(\C)CC/C=C(\C)CCC=C(C)C]

Medium-chain triglycerides (MCTs) are medium-chain (6 to 12 carbons) fatty acid esters of glycerol. Coconut oil is composed of approximately 66% medium-chain triglycerides. Other rich sources of MCTs include palm kernel oil and camphor tree drupes. The fatty acids found in MCTs are called medium-chain fatty acids. The names of the medium-chain fatty acids (and the corresponding number of carbons) found in MCTs are caproic acid (C₆), caprylic acid (C₈), capric acid (C₁₀) and lauric acid (C₁₂). MCTs are composed of a glycerol backbone and three of these fatty acids. The approximate ratios of these fatty acids in commercial MCT products derived from coconut oil are 2(C₆):55(C₈):42(C₁₀):1(C₁₂).

MCTs are also known as 1,2,3-Propanetriol Trioctanoate, AC-1202, Capric Acid, Caproic Acid, Caprylic Acid, Caprylic Triglycerides, Lauric Acid, MCT, MCT's, Medium-Chain Triacylglycerols, Medium-Chain Triglycerides, Tricaprylin, Triglycérides à Chaîne Moyenne, Triglicéridos de Cadena Media (TCMs), and Trioctanoin.

Triglycerides are a form of fat found in food and in the body. There are four main types of triglycerides: long-chain fatty acids, very long-chain fatty acids, medium-chain fatty acids and short-chain fatty acids. Long-chain triglycerides contain 16-24 carbon atoms. Medium-chain triglycerides contain 8-12 carbons. Short-chain fatty acids contain less than 8 carbon atoms. Very long-chain triglycerides contain over 24 carbon atoms. Most common fats and oils contain long-chain fatty acids.

Medium chain triglycerides (MCTs) are partially man-made fats. The name refers to the way the carbon atoms are arranged in their chemical structure. MCTs are generally made by processing coconut and palm kernel oils in the laboratory.

Soybean oil is a vegetable oil extracted from the seeds of the soybean (Glycine max), and is used in cosmetics as an emollient. Soybean oil contains 61% polyunsaturated fat and 24% monounsaturated fat, and consists of mostly glycerides of linoleic, oleic, linolenic and plamitic acids. Super refined soybean oil has been purified to remove impurities. While conventional soybean oil is pale yellow in appearance, super refined soybean oil is a water-white, odourless, neutral non-polar lipid with a low peroxide value. Preferably, super refined soybean oil has less than about 10% impurities, less than about 9% impurities, less than about 8% impurities, less than about 7% impurities, less than about 6% impurities, less than about 5% impurities, less than about 4% impurities, less than about 3% impurities, less than about 2% impurities, less than about 1% impurities, less than about 0.5% impurities, less than about 0.1% impurities, 0.05% impurities, or 0.01% impurities. Examples of commercially available super refined soybean oil include Cropure™ Soybean, Super Refined™ Soybean, Super Refined™ Soybean EP NP, Super Refined™ Soybean USP, Super Refined™ Soybean USP NP, and Super Refined™ Soybean USP JP NP.

4. Aqueous Phase

The aqueous phase can be water or a buffer, including a physiologically compatible solution such as water or phosphate buffered saline.

5. Active Agents

In a further embodiment, the hormonal nano formulation additionally comprises one or more non-hormone active agents. The presence of such a non-hormone active agent is not required, and substantial and unexpected anti-aging and/or anti-wrinkle properties are observed without the presence of such a non-hormone active agent. Thus, the presence of such one or more non-hormone active agents is to merely enhance the anti-aging and anti-wrinkle properties of the hormonal nano formulations of the invention. In another embodiment, the hormonal nano formulation further comprises at least one non-hormone active agent for rendering the composition suitable as a cosmetic preparation.

In one embodiment, active agents useful in the hormonal nano formulations of the invention are compounds that provide benefits to the skin and/or provide desirable properties to a composition formulated as a cosmetic or medicinal preparation. The non-hormone active agent useful in the hormone emulsion compositions of the invention can be a drug substance or a non-drug substance. Examples of non-drug active agents include, but are not limited to, skin lightening agents, tanning agents, skin conditioning agents, skin protectants, emollients and humectants, and sunscreen actives.

Examples of exemplary non-hormone active agents useful in the hormonal nano formulations of the invention, such as a non-hormone active drug substance or an active cosmetic substance, include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids), colloidal oatmeal (Aveeno®), soybean extract, or any mixture thereof.

Representative sunscreen drugs are active ingredients that absorb, reflect, or scatter radiation in the UV range at wavelengths from 290 to 400 nanometers. Specific examples include benzophenone-3 (oxybenzone), benzophenone-4 (sulisobenzone), benzophenone-8 (dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)]aminobenzoate), ethylhexyl dimethyl PABA (Padimate 0), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate (trolamine salicylate), titanium dioxide, and zinc oxide. One skilled in the art will appreciate that other sunscreen agents may be used in the compositions and preparations of the present invention.

As noted above, the hormonal nano formulations of the invention may further comprise skin conditioning agents. Such agents comprise substances that enhance the appearance of dry or damaged skin, as well as materials that adhere to the skin to reduce flaking, restore suppleness, and generally improve the appearance of skin. Representative examples of skin conditioning agents include acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and derivatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples of skin conditioning agents include, but are not limited to, lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanbl, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. Skin conditioning agents, other than those listed above, may also be used, as is readily appreciated by those skilled in the art.

Examples of topical steroids, antifungal agents, antiviral agents, topical anesthetic agents, and NSAIDS that can be utilized in the nano hormonal formulations of the invention are given above.

6. Additional Ingredients

Additional compounds suitable for use in the hormonal nano formulations of the invention include but are not limited to bulking agents, viscosity promoting agents, coloring agents, pharmaceutically acceptable excipients, antioxidants, microbial preservatives, perfume, pH adjusters, buffers, etc.

Suitable antioxidants and microbial preservatives for use in the hormonal nano formulations of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof.

The hormonal nano formulations may further comprise at least one pH adjuster. Suitable pH adjusters in the emulsions of the invention include, but are not limited to, diethyanolamine, lactic acid, citric acid, hydrochloric acid, monoethanolamine, triethylanolamine, sodium hydroxide, sodium phosphate, semi-synthetic derivatives thereof, and combinations thereof.

The hormonal nano formulation can comprise a buffering agent, such as a pharmaceutically acceptable buffering agent. In addition, the hormonal nano formulation can comprise one or more emulsifying agents or surfactants to aid in the formation of emulsions. Emulsifying agents include compounds that aggregate at the oil/water interface to form a kind of continuous membrane that prevents direct contact between two adjacent droplets.

In addition to the foregoing active agents, the hormonal nano formulations employed in the methods of the present invention may also comprise inert and physiologically acceptable carriers or diluents.

7. Solvent

The formulations of the invention can optionally comprise one or more solvents. Any suitable solvent can be used in the methods and formulations of the invention. Exemplary solvents include, but are not limited, to C₁-C₁₂ alcohols, isopropyl myristate, triacetin, N-methylpyrrolidinone, aliphatic or aromatic alcohols, polyethylene glycols, ethanol, and propylene glycol. An example of an alcohol useful in the present invention includes, but is not limited to ethanol. Other short chain alcohols and/or amides may be used. Other solvents include dimethyl sulfoxide, dimethyl acetamide, and ethoxydiglycol. Mixtures of solvents can also be used in the compositions and methods of the invention.

B. Emulsion Droplet Size

The hormonal nano formulations of the invention comprise emulsion droplets having an average diameter of less than about 10 microns to about 1 nm. In other embodiments of the invention, the emulsion droplets can have an average diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In one embodiment, the hormonal nano formulation droplets have an average diameter of less than about 2 microns, with a mean diameter of about 1 micron preferred. In another embodiment of the invention, the droplets are filterable through a 5 micron filter, a 2 micron filter, a 0.45 micron filter, or a 0.2 micron filter.

The hormonal nano formulation globules can be made using food grade, USP or NF grade materials suitable for human use applications.

C. Stability of the Hormonal Nano Formulations

The hormonal nano formulations of the invention can be stable when exposed to room temperature (e.g., about 25° C.) and up to about 50° C. In other embodiments, the hormonal nano formulations of the invention are stable when exposed to a temperature selected from the group consisting of greater than about 50° C., about 55° C. or greater than about 55° C., about 60° C. or greater than about 60° C., about 65° C. or greater than about 65° C., about 70° C. or greater than about 70° C., about 75° C. or greater than about 75° C., about 80° C. or greater than about 80° C., or about 85° C. or greater than about 85° C.

In addition, the hormonal nano formulations of the invention can be stable when exposed to an elevated temperature of 50° C. for a duration of 1 month and room temperature storage of 25° C. for up to 3 years.

Exemplary thermostable surfactants and/or stabilizers include, but are not limited to, (1) sorbitan esters, such as Spans and Arlacel, (2) block polymers, such as Pluronics, (3) acrylic polymers, such as Pemulen, and (4) ethoxylated fatty esters, such as Cremophor RH-40.

D. Pharmaceutical Compositions

The hormonal nano formulations of the invention may be formulated into pharmaceutical compositions that comprise the hormone in a therapeutically effective amount and suitable, pharmaceutically-acceptable excipients for topical administration to a human subject in need thereof. Such excipients are well known in the art.

By the phrase “therapeutically effective amount” it is meant any amount of the hormone that is effective in preventing and/or treating the age-related condition. Topical administration includes administration to the skin.

Pharmaceutically acceptable dosage forms for topical administration include, but are not limited to, ointments, creams, liquids, emulsions, lotions, gels, bioadhesive gels, aerosols, pastes, foams, or in the form of an article or carrier, such as a bandage, insert, powder, talc or other solid.

The pharmaceutical hormonal nano formulations of the invention may be formulated for immediate release, sustained release, controlled release, delayed release, or any combinations thereof, into the epidermis or dermis, with minimum to no systemic absorption.

In some embodiments, the hormonal nano formulation is formulated for delivery via a “patch” comprising a therapeutically effective amount of the hormone.

The pharmaceutical hormonal nano formulation may be applied in a single administration or in multiple administrations. The pharmaceutical hormonal nano formulation can be applied for any suitable time period in one or more applications per day. For example, the hormonal nano formulation can be topically applied for at least once a week, at least twice a week, at least once a day, at least twice a day, multiple times daily, multiple times weekly, biweekly or any combination thereof. The pharmaceutical hormonal nano formulation can be topically applied for a period of time of about one week to about 5 years or as long as it is necessary to maintain healthy skin and as prescribed by a physician. Between applications, the application area may be washed to remove any residual hormonal nano formulation.

Preferably, the pharmaceutical hormonal nano formulations are applied to the skin area in an amount of from about 0.001 mL/cm² to about 1.0 mL/cm². An exemplary application amount and area is about 0.2 mL/cm², although any amount from 0.001 mL/cm² up to about 1.0 mL/cm² can be applied. Following topical administration, the hormonal nano formulations may be occluded or semi-occluded. Occlusion or semi-occlusion may be performed by overlaying a bandage, polyoleofin film, impermeable barrier, or semi-impermeable barrier to the topical preparation.

Typically, for a method of the present invention, aside from the content of the hormonal nano formulation used a small amount of the composition (from about 1 ml to about 5 ml) is applied to exposed areas of skin from a suitable container or applicator, and, if necessary, the hormonal nano formulation is then spread over and/or rubbed into the skin using the hand, finger, or other suitable device. Each hormonal nano formulation disclosed herein is typically packaged in a container that is appropriate in view of the hormonal nano formulation's viscosity and intended use by the consumer. For example, a lotion or fluid cream may be packaged in a bottle, roll-ball applicator, capsule, propellant-driven aerosol device, or a container fitted with a manually operated pump. A cream may simply be stored in a non-deformable bottle, or in a squeeze container, such as a tube or a lidded jar. A unit dose can be a packaged in a disposable pouch for application.

### IV. Methods of Making the Hormonal Nano Formulations

The hormonal nano formulations of the invention are made using pharmaceutically acceptable homogenization techniques. One benefit of the methods of making the compositions to be utilized in the pharmaceutical dosage forms of the invention as compared to prior art methods, such as wet milling, is that the methods are applicable to water-soluble hormones and non-hormone active agents as well as poorly water-soluble hormones and non-hormone active agents. Another benefit of the methods of the invention is that they do not require grinding media or specialized grinding process or equipments. The use of such grinding media can add cost and complexity to a manufacturing method. Additionally, unlike wet milling technologies, the methods of the invention can accommodate amorphous or semi-amorphous hormones.

For example, a homogenizing process can comprise passing a hormonal formulation of the invention through a high-pressure homogenization system at 1,000 to 10,000 psi until the desired particle (droplet) size is obtained. Additional passes through the homogenizer can be utilized as needed.

In an exemplary method, the MCT and/or squalene (and/or squalane) are combined with the at least one surfactant, and the hormone is then solubilized in the resultant mixture. The aqueous phase (e.g., water or buffer) is then added to the hormone/MCT/surfactant mixture (or hormone/squalene(squlane)/surfactant mixture). The resultant composition is then subjected to relatively high pressure using the homogenizer to obtain a suitable hormonal nano formulation The discontinuous phase of MCT/surfactant (or squalene/surfactant) droplets having an average diameter of less than about 10 microns dispersed in an aqueous continuous phase, such as buffer or water, is produced using this process.

A hormonal nano formulation according to the invention can also be produced with reciprocating syringe instrumentation, continuous flow instrumentation, or high speed mixing equipment. High velocity homogenization or vigorous stirring, producing forces of high shear and cavitation, are preferred. High shear processes are preferred as low shear processes can result in larger droplet sizes.

The hormonal nano formulations of the invention are stable for many months at a broad range of temperatures, and do not decompose even after long storage periods at room temperature (e.g., about 25° C.) for up to about 3 years. In addition, the hormonal nano formulations of the invention can be produced in large quantities using commercial equipment and homogenizers.

If desired, the water miscible droplets can be filtered through a suitable filter, such as a 5 micron filter, a 2 micron filter, a 0.45 micron filter, or a 0.2 micron filter.

### V. Methods of Using the Nano Formulations of the Invention

The hormonal nano formulations of the invention are administered to a subject topically via any conventional means. As used herein, the term “subject” is used to mean an animal, preferably a mammal, including a human or non-human. The terms patient and subject may be used interchangeably. In addition, the hormonal nano formulations of the invention can be formulated into any suitable dosage form. Moreover, the dosage forms of the invention may be solid dosage forms, liquid dosage forms, semi-liquid dosage forms, immediate release formulations, modified release formulations, controlled release formulations, delayed release formulations, extended release formulations, pulsatile release formulations, and mixed immediate release and controlled release formulations, or any combination thereof.

Dosage unit compositions may comprise such amounts or submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient. The duration of the treatment; drugs used in combination or coincidental with the specific agent; and like factors well known in the medical arts.

The methods of the invention comprise topically applying a hormonal nano formulation according to the invention to treat any age-related condition. For example, signs of aging skin and/or skin imperfections which can be treated, prevented, and/or minimized with the methods of the invention include, but are not limited to, (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content, (10) melasmas (atypical pigmentation or hyper-pigmentation of the skin), (11) skin clarity and/or radiance, (12) skin smoothness and/or softness, (13) pore size (larger pore can make an individual appear older), (14) increase hydration, (15) increase skin smoothness, (16) increase skin tightness, (17) oral and/or genital herpes lesions, (18) skin wounds, (19) skin infections, and any combination thereof. The compositions of the invention can also be used to treat scars, such as acne and chickenpox scars. Collectively the signs of aging skin, skin imperfections and scars are referred to as “dermatological conditions.”

In one embodiment of the invention, the hormonal nano formulations of the invention produce a visible difference in fine lines and wrinkles For example, the visible difference can be about a 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about a 100% decrease in fine lines and wrinkles.

In another embodiment of the invention, the hormonal nano formulations of the invention produce an increase in stem cell hair follicles. For example, the visible difference can be about a 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about a 100% increase in stem cell hair follicles.

In another embodiment of the invention, the hormonal nano formulations of the invention produce a visible difference in a dermatological conditions. For example, the visible difference can be about a 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about a 100% decrease in fine lines and wrinkles.

### VI. Examples

The invention is further described by reference to the following examples, which are provided for illustration only. The invention is not limited to the examples, but rather includes all variations that are evident from the teachings provided herein. All publicly available documents referenced herein, including but not limited to U.S. patents and U.S. patent publications, are specifically incorporated by reference.

### Example 1

The purpose of this example was to prepare two estrogen nano formulations for testing and evaluation.

The components of the first composition (Formulation 1) included the hormone estradiol, the poloxamer Pluronic F68, medium chain triglyceride, and purified water and are described in Table 3 below. The particle size of the resultant formulation was about 10 microns.

TABLE 3
Formulations 1A, 1B, and 1C

| | High Dose | Mid Dose | Low Dose |
|---|---|---|---|
| Ingredient | Estradiol | Estradiol | Estradiol |
| Estradiol | 250 mg | 25 mg | 5 mg |
| Pluronic F 68 | 6 g | 6 g | 6 g |
| Medium Chain Trigyceride | 47 g | 47 g | 47 g |
| Purified Water | 46.75 g | 46.95 g | 46.995 g |

Manufacturing procedure: The Medium Chain Triglyceride was weighed and placed in a suitable glass vessel #1. Next, the Pluronic F-68 (poloxamer) was weighed and added to the glass vessel #1 with gentle mixing. The estradiol was then weighed and added to the glass vessel #1 with gentle mixing until dispersed. Finally, the Purified Water was weighed and added to the glass vessel #1 with moderate mixing.

The mixture from glass vessel #1 was then passed through a high pressure homogenizer (APV 1000 Homogenizer, SPX Corp.) operating at 10,000 psi until an emulsion was formed. This process was repeated by homogenizing the emulsion under the same setting.

A second emulsion composition (Formulation 2) was made as described above, with the medium chain triglyceride replaced with squalene as detailed in Table 4 below. The particle size of the resultant formulation was about 10 microns.

TABLE 4
Formulations 2A, 2B, and 2C

| | High Dose | Mid Dose | Low Dose |
|---|---|---|---|
| Ingredient | Estradiol | Estradiol | Estradiol |
| Estradiol | 250 mg | 25 mg | 5 mg |
| Pluronic F68 | 5 g | 5 g | 5 g |
| Squalene | 52 g | 52 g | 52 g |
| Purified Water | 42.75 g | 42,975 g | 42.995 g |

### Example 2

The purpose of this example was to evaluate and compare the compositions of Example 1 and the commercially available transdermal estrogen formulation, Estrasorb®, to determine the effectiveness of the hormonal nano formulations of the invention as a trans-epidermal formulation providing the histological benefits of a hormone such as estrogen without the risks associated with systemic estrogen absorption.

Estrasorb® (estradiol topical emulsion) is designed to deliver estradiol to the blood circulation following topical application of an emulsion. See http://www.rxlist.com/estrasorb-drug.htm (accessed on Jan. 27, 2012). Each gram of Estrasorb® contains 2.5 mg of estradiol hemihydrate USP, EP, which is encapsulated using a micellar nanoparticle technology. Estrasorb® is packaged in foil pouches containing 1.74 grams of drug product. Daily topical application of the contents of two foil pouches provides systemic delivery of 0.05 mg of estradiol per day. Estradiol hemihydrate USP, EP (estradiol) is a white, crystalline powder, chemically described as (17β)-estra-1,3,5(10)-triene-3,17-diol, hemihydrate. The molecular formula of estradiol hemihydrate is C₁₈H₂₄O₂, ½ H₂O, and the molecular weight is 281.4 g/mol. The structural formula of estradiol is:

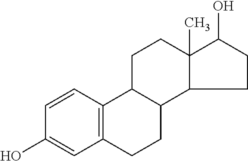 [SMILES: CC12CCC3c4ccc(O)cc4CCC3C1CCC2O]

The active ingredient in Estrasorb is estradiol. The remaining components (soybean oil, water, polysorbate 80, and ethanol) are pharmacologically inactive.

Estrogen is the term for a class of different hormones. Estradiol is one type of estrogen (humans have more than 25 different types of estrogen). Estradiol is the most common estrogen in women of childbearing age.

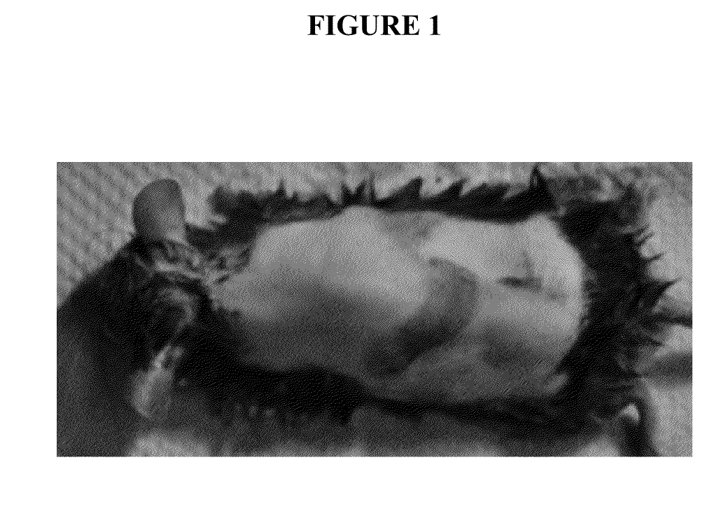

A total of 15 male mice aged approximately 1 year were treated with topical estrogen for 21 days at a time. The mice were then exposed to isofluorane in a sealed container for 15 seconds, after which open exposure to isofluorane was used to maintain an anesthetic state. Calcium/sodium hydroxide topical hair removal cream was applied for 3-4 minutes and subsequently washed off, exposing hairless skin. See FIG. 1.

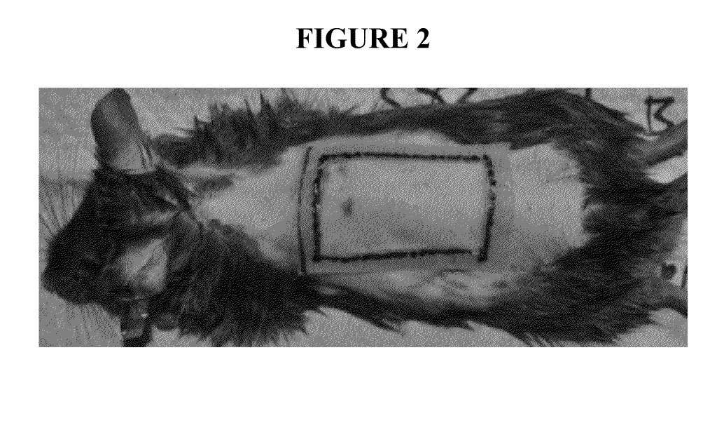

Pursuant to the instructions provided in the Estrosorb® pharmacological information packet and clinical application practice, a 1.5/2.0 cm area of skin was isolated on the dorsum of the mouse, below the neck. See FIG. 2. Topical estrogen/exposure area was estimated by calculating the average percent of skin attributed to anterior thigh (recommended treatment area for women undergoing HRT via Estrasorb®), and applying like amounts to the 1.5/2.0 cm area of the mouse (Sacco et. al., “The Average Body Surface Area of Adult Cancer Patients in the UK: A Multicentre Retrospective Study,” PLoS ONE, 5 (1): e8933 (2010); doi:10.1371/journal.pone.0008933, which summarizes statistical studies reviewing the average body surface area of women over 60).

Estrogen Application:

The 15 mice were placed into the following groups:
(1) Group 1: Estrasorb® (0.25% estradiol) treatment (n=1);
(2) Group 2: treatment with squalene high dose estradiol (0.25% estradiol) nano formulation (n=4);
(3) Group 3: treatment with squalene low dose estradiol (0.005%% estradiol) nano formulation (n=2);
(4) Group 4: treatment with squalene placebo, containing no estradiol (n=4);
(5) Group 5: treatment with MCT-mid dose estradiol (0.025% estradiol) nano formulation (n=2); and
(6) Group 6: treatment with MCT-placebo, containing no estradiol (n=2).

Each mouse was swabbed daily on the duoderm and the amount of the composition applied was recorded (average of 30 mg daily, SD=5 mg). The mice were treated for 20-60 days (60 days of treatment for latest batch as per time needed for steroid-skin modulation) (Ehrhardt et al., “The effects of 20-hydroxyecdysone estradiol on the skin of ovariectomized rats,” Menopause, 18(3):323-7 (2011), provides a histological analysis of rats after 12 weeks of topical estrogen treatment).

TABLE 5

| | | Group 2 | Group 3 | Group 4 | Group 5 | Group 6 |
|---|---|---|---|---|---|---|
| | Group I | Squalene/high | Squalene/low | Squalene | MCT/mid | MCT- |
| | Estrasorb ® | dose estradiol | dose estradiol | placebo | dose estradiol | placebo |
| Days | 60 | 60 | 21 | 60 | 21 | 21 |
| applied | | | | | | |

Histology:

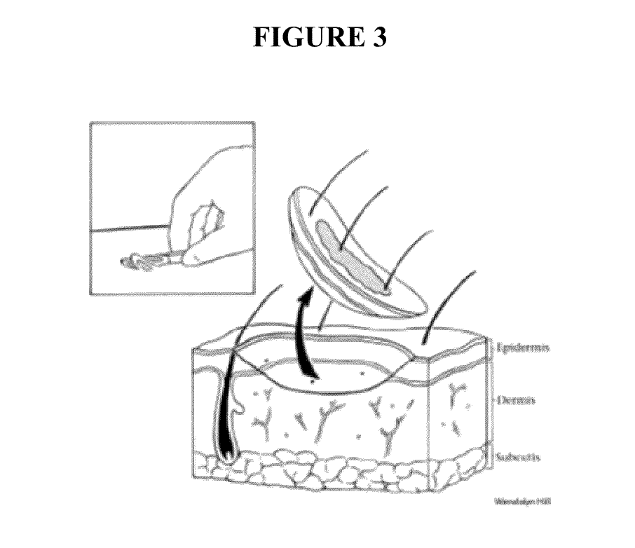

Samples were harvested on day 20-60, depending on the sample group (see Table 5). Samples were excised using Metzenbaum curved scissors (FIG. 3) and fixed in formalin for 24 hours. Samples were then taken directly to an independent histology lab for HE analysis. Blank samples were requested for subsequent picosirius red staining and evaluation under polarized light.

Serum Studies:

Isofluorane anesthesia with jugular venous puncture were used for blood harvesting. Specimens were exsanguinated and blood was allowed to stand at room temp for 30 min. Samples were then centrifuged at 1300 RPM for 20 minutes and the plasma layer removed and stored in −20° C.

Plasma Preparation:

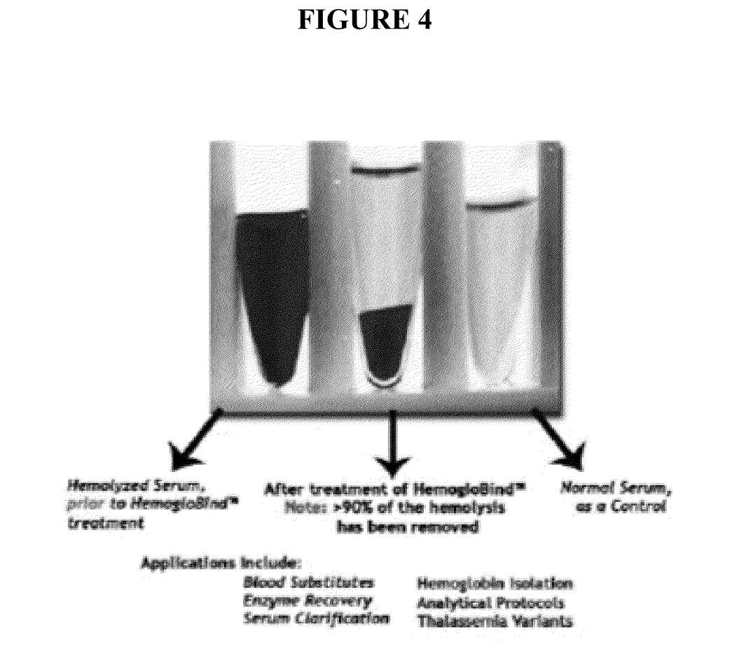

Due to imperfect blood harvesting techniques, red blood cells (RBC) are often lysed, spilling hemoglobin in to the plasma. This results in pinkred appearing plasma. Treatment with copper/cobalt beads binds to the his-residues found on hemoglobin thus isolating hemoglobin free plasma. See FIG. 4, which shows hemolyzed serum prior to HemogloBind™ treatment, after treatment with HemogloBind™ where >90% of the hemolysis has been removed, and normal serum as a control.

Plasma Analysis:

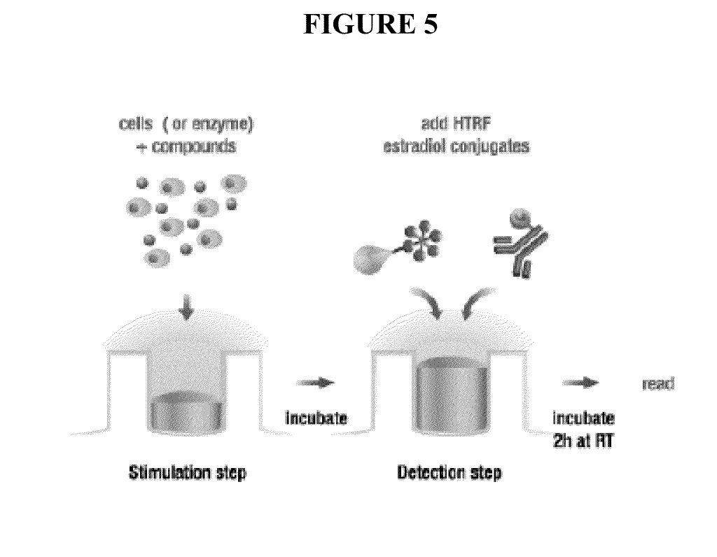

A Cayman Chemical estradiol ELISA kit was used for the plasma analysis. Similar assays have been done at MIT when evaluating serum estradiol and its effects on gastric cancer (Sheh et al., “17-beta estradiol and Tomoxifen Prevent Gastric cancer by Modulating Leukocyte Recruitment and Oncogenic Pathways in Helicobacter Pylori-Infected INS-GAS Male Mice,” Cancer Prev. Res., 4:1426-1435 (2011)). A 5:1 dilution was used as recommended. All samples were evaluated after maxbinding reached a minimum of 0.3 units. See FIG. 5.

Results: Estrosorb.

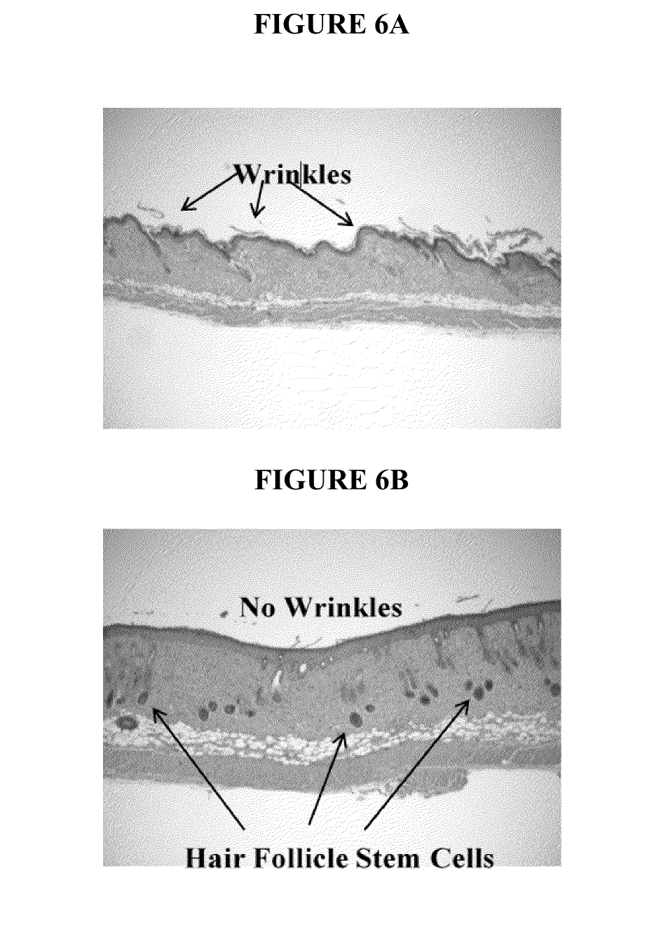

Dilutions of 1/100 (0.0025% estradiol) were made by the addition of PBS (Phosphate buffered saline)-based placebo solution to full strength (0.25% w/w) estrasorb. The diluted Estrasorb® was applied for 21 days. The H/E histology was analyzed and showed that both formulations (0.0025% estradiol and 0.25% estradiol) minimized wrinkles, thickened the epidermis, and increased stem cell hair follicles seen. FIG. 6A shows a cross section of mouse skin with no treatment, with wrinkles in the skin clearly visible. In sharp contrast, FIG. 6B shows a cross section of mouse skin following treatment with the diluted Estrasorb® (0.0025% estradiol), with the absence of wrinkles clearly evident. In addition, the diluted Estrasorb® (0.0025% estradiol)-treated mouse skin also clearly shows the evidence of hair follicle stem cells, which are absent in the un-treated skin.

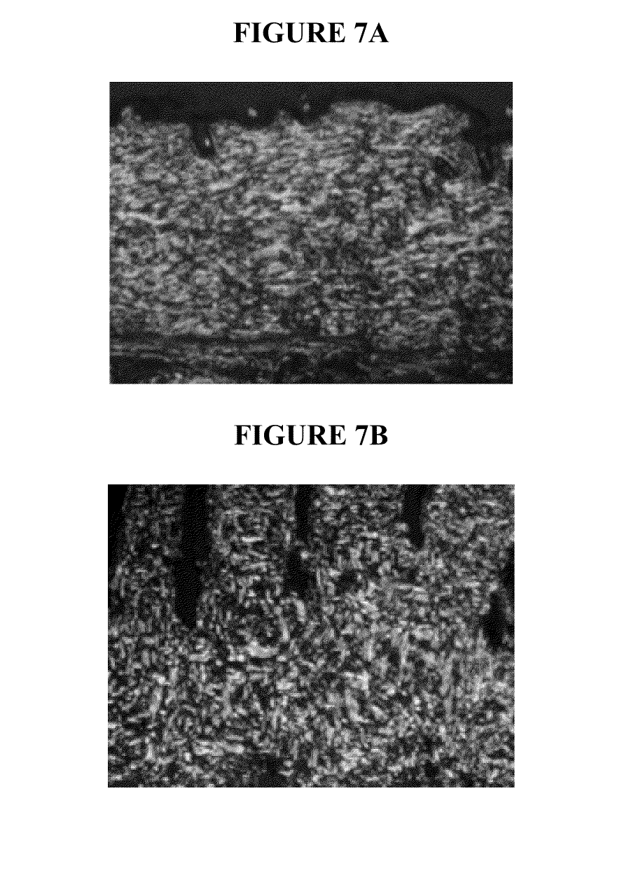

Tissue samples of the Estrasorb® treated skin of 0.25% estradiol (treated for 60 days) were also subjected to picosirius red stains to analyze the samples for collagen packing and density. The results, shown in FIGS. 7A and 7B are dramatic. FIG. 7A shows picosirius red stain of a mouse skin sample in the absence of Estrasorb® treatment and FIG. 7B shows picosirius red stain of a mouse skin sample following Estrasorb® treatment. The tightly packed and better aligned collagen molecules present in FIG. 7B show polarization colors of longer wavelengths (yellow-red). See Dayan et al., “Are the polarization colors of picosirius red-stained collagen determined only by the diameter of the fibers?”Histochemistry, 93(1):27-9 (1989).

Squalene-Estrodiol Results:

Next, histology results were evaluated for the squalene low dose estradiol (0.005%% w/w estradiol) nano formulation as compared to a squalene control formulation. The results are shown in FIGS. 8A (squalene control) and 8B (0.005% w/w estradiol squalene nano formulation). A cross section of mouse skin shown in FIG. 8A exhibits a thin epidermis. In contrast, the cross section of mouse skin shown in FIG. 8B exhibits a thicker epidermis and evidence of hair follicle stem cells.

Thus, the low dose estradiol (0.005% w/w estradiol) squalene nano formulation showed significant dermal thickening with what appear to be reduced wrinkles when compared to both squalene placebo (FIG. 8A) and pre treatment controls (FIG. 6A).

In addition, these effects were local, as samples taken from other areas of the treatment animal showed similar results as placebo, without increased dermal thickening and reduction in wrinkles. Surprisingly, this localization-effect was not seen in the Estrasorb® treatment group, where dermal thickening and reduction in wrinkles were seen both on treated skin and non-treated skin. Such results are likely the product of the systemic absorption of estradiol from the Estrasorb® formulation.

Tissue samples of skin treated with squalene low dose estradiol (0.005% estradiol) nano formulation were subjected to picosirius red stains to analyze the samples for collagen packing and density. The results, shown in FIGS. 9A and 9B are dramatic. FIG. 9A shows picosirius red stain of a mouse skin sample in the absence of treatment and FIG. 9B shows picosirius red stain of a mouse skin sample following treatment with squalene low dose estradiol (0.005% estradiol) nano formulation. The tightly packed and better aligned collagen molecules present in FIG. 9B show polarization colors of longer wavelengths (yellow-red). See Dayan et al., Histochemistry, 93(1):27-9 (1989).

Tissue samples of skin treated with squalene high dose estradiol (0.25% estradiol) nano formulation treated skin were subjected to picosirius red stains to analyze the samples for collagen packing and density. The results, shown in FIGS. 10A and 10B are dramatic. FIG. 10A shows picosirius red stain of a mouse skin sample in the absence of treatment and FIG. 10B shows picosirius red stain of a mouse skin sample following treatment with squalene high dose estradiol (0.25% estradiol) nano formulation. The tightly packed and better aligned collagen molecules present in FIG. 10B show polarization colors of longer wavelengths (yellow-red). See Dayan et al., Histochemistry, 93(1):27-9 (1989).

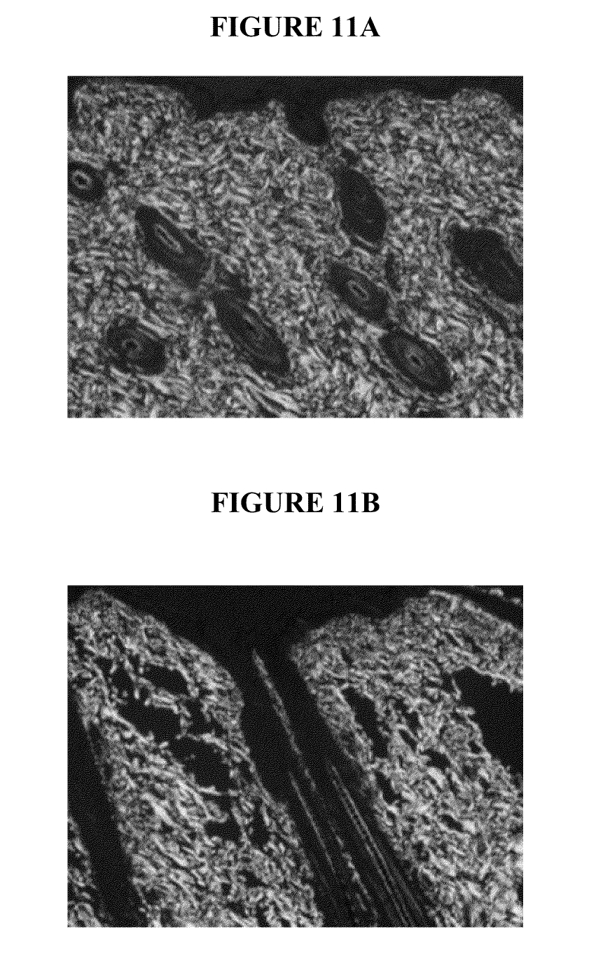

As a comparison, tissue samples of skin treated with squalene placebo were subjected to picosirius red stains to analyze the samples for collagen packing and density. The results are shown in FIGS. 11A and 11B. FIG. 11A shows picosirius red stain of a mouse skin sample in the absence of treatment and FIG. 11B shows picosirius red stain of a mouse skin sample following treatment with squalene placebo. Post treatment shows no difference in the density of the collagen molecules, and FIG. 11B appears to show polarization colors of shorter wavelengths, indicating a decrease in collagen fibers.

Squalene Serum Estradiol Levels:

serum from the following test groups was evaluated for serum estradiol levels after 60 days. Estrasorb®, squalene high dose estradiol nano formulation, squalene placebo, squalene low dose estradiol nano formulation, MCT mid-dose (0.025%) estradiol nano formulation, and MCT placebo. See FIG. 12. The results are summarized in Table 6 below.

TABLE 6

| | | Group 2 | Group 3 | | Group 5 | |
|---|---|---|---|---|---|---|
| | | Squalene/ | Squalene/low | Group 4 | MCT/mid | Group 6 |
| | Group I | high dose | dose | Squalene | dose | MCT- |
| | Estrasorb ® | estradiol | estradiol | placebo | estradiol | placebo |
| Estradiol | 190 | 20 | 5 | 18 | 19 | 3 |
| level | | | | | | |
| (pg/mL) | | | | | | |

As expected, Estrasorb®, which is designed to delivery estradiol systemically, exhibited very high levels of serum estradiol. However, the other results shown in FIG. 12 were highly unexpected. Notably, the squalene low dose estradiol nano formulation exhibited a lower serum estradiol level than the squalane placebo composition! (This is possible as estradiol is naturally present in the blood stream.) Moreover, all of the hormonal nano formulations tested according to the invention, namely (1) the squalene high dose estradiol nano formulation, (2) the squalene low dose estradiol nano formulation; and (3) the MCT-mid dose estradiol nano formulation, exhibited minimal serum levels of estradiol, which were about the same or less than the serum estradiol levels measured for the squalene placebo composition. See FIG. 12.

These results are exceptional as while the hormonal nano formulations according to the invention exhibited minimal or no systemic absorption of estradiol, they also exhibited significant localized effects in terms of thickening the epidermis (FIG. 8B; 0.25% estradiol squalene nano formulation), stimulating the production of hair follicle stem cells (FIG. 8B; 0.25% estradiol squalene nano formulation), and stimulating the production of collagen molecules (FIG. 9B; 0.025% estradiol squalene nano formulation and FIG. 10B; 0.25% estradiol squalene nano formulation).

Results:

The results of this experiments demonstrate that estradiol topically applied to the skin results in increased dermal thickness, reduced wrinkles, and increased and more organized collagen. Moreover, the results also demonstrate that a topically applied squalene-estrogen nano formulation according to the invention yields similar histological results, with a decrease in wrinkles, thickened epidermis and increased stem cell hair follicles. However, while the Estrasorb® application led to thickening of skin at the site of application and elsewhere (though to a lesser extent), the effects of the squalene estradiol nano formulation, e.g., skin thickening, was localized to the site of application. Furthermore, exposure of skin to both Estrasorb® and the squalene-estradiol nano formulation resulted in more densely packed collagen fibers.

Finally, the results demonstrate that treatment with a topical squalene estrogen nano formulation according to the invention does not result in significant systemic absorption of the component hormone, e.g., estradiol. Specifically, the data shows that there appears to be little if any systemic absorption of estradiol via topical application of a squalene estradiol nano formulation according to the invention, as determined by plasma estradiol concentration determined by ELISA assay. In contrast, topical application of Estrasorb® results in dramatic increases in serum estradiol.

### Example 3

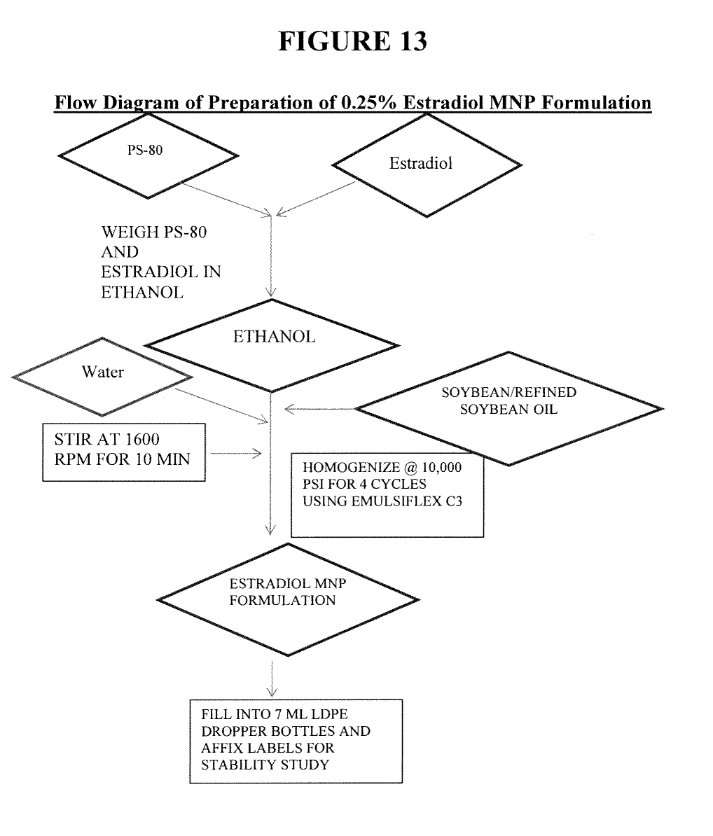

The purpose of this example is to describe an exemplary method of making a nanoemulsion (MNP) formulation comprising refined soybean oil, where the formulation is useful as a delivery vehicle for hormones, either alone or in combination with a non-hormone active agent. The method is shown in a flow chart depicted in FIG. 13, which depicts preparation of formulation additionally comprising the active estradiol.

An accurate quantity of ethanol is first weighed into a 500 mL beaker. Next, estradiol is weighed and transferred into the 500 mL beaker, followed by sonication to dissolve properly. Next, Polysorbate 80 is weighed and transferred into the 500 mL beaker followed by mixing well. Finally, super refined soybean oil is weighed and transferred into the 500 mL beaker followed by mixing well. The contents of the beaker is then mixed for 10 minutes with the addition of a weighed quantity of purified water slowly at 1600 r.p.m using a propeller stirrer. Finally, the mixture is passed through a high pressure homogenizer at 10,000 to 12,000 psi, and repeated for a total of 4 homogenization cycles. Homogenization time: 6 min/cycle for 0.25 L batch.

Filling and labeling: the placebo MNP formulation is then filled into 7 ml LDPE dropper bottles, and labeled them for accelerated stability studies at 40° C./75% RH and room temperature.

### Example 4

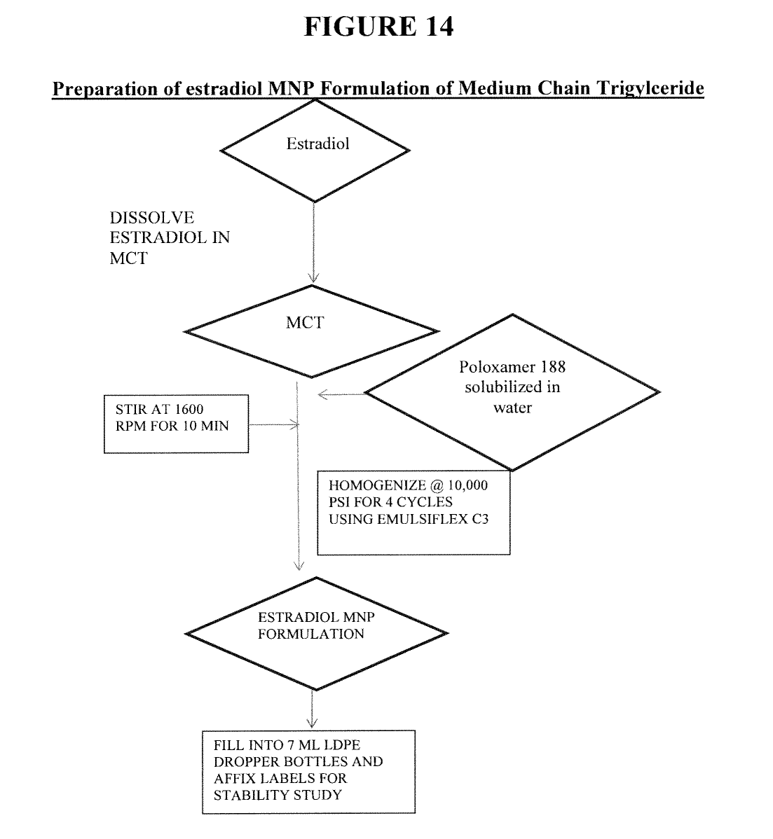

The purpose of this example is to describe an exemplary method of making a nanoemulsion (MNP) formulation comprising a medium chain triglyceride (MCT), where the formulation is useful as a delivery vehicle for hormones, either alone or in combination with a non-hormone active agent. The method is shown in a flow chart depicted in FIG. 14, which depicts preparation of formulation additionally comprising the active estradiol.

An accurate quantity of purified water is first weighed into a 500 mL beaker. Next, poloxamer 188 (Pluronic F68) is weighed and transferred into a the beaker, followed by placing the beaker on a magnetic stirrer until the poloxamer is fully solubilized. Next, a medium chain triglyceride (Miglyol® 812) is weighed and transferred into a second beaker. The contents of the second beaker is then mixed for 10 min. with the addition of the poloxamer 188 (Pluronic F68) solution from the first beaker slowly added at 1600 r.p.m using a propeller stirrer. The contents of the resultant mixture are passed through a high pressure homogenizer at 10,000 to 12,000 psi, for a total of 4 homogenization cycles.

Filling and labeling: the placebo MNP formulation is then filled into 7 ml LDPE dropper bottles, and labeled them for accelerated stability studies at 40° C./75% RH and room temperature.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A hormonal nano formulation comprising:
(a) at least one hormone;
(b) at least one surfactant;
(c) water; and
(d) squalene, squalane, super refined soybean oil, a medium chain triglyceride, or any combination thereof,
wherein the formulation comprises droplets having an average diameter of less than about 10 microns.

2. The formulation of claim 1 comprising squalene or squalane.

3. The formulation of claim 1 comprising a medium chain triglyceride.

4. The formulation of claim 1 comprising super refined soybean oil.

5. The formulation of claim 1, wherein the at least one hormone is synthetic or naturally occurring.

6. The formulation of claim 1, wherein the at least one hormone is selected from the group consisting of estrogens, progestagens, estrone, estradiol, genestein, estriol, equilin, equilenin, chlorotrianisene, dienestrol, diethylstilbestrol, ethinylestradiol, fosfestrol, mestranol, polyestradiol phosphate, xenoestrogens, phytoestrogens, mycoestrogens, progestagens, progesterone, desogestrel, drospirenone, dydrogesterone, ethisterone, etonogestrel, ethynodiol diacetate, gestodene, gestonorone, levonorgestrel, lynestrenol, medroxyprogesterone, megestrol, norelgestromin, norethisterone, norethynodrel, norgestimate, norgestrel, norgestrienone, tibolone, melatonin and testosterone.

7. The formulation of claim 1, wherein the at least one surfactant is selected from the group consisting of pharmaceutically acceptable ionic surfactants, pharmaceutically acceptable non-ionic surfactants, pharmaceutically acceptable anionic surfactants, pharmaceutically acceptable cationic surfactants, and pharmaceutically acceptable zwitterionic surfactants.

8. The formulation of claim 1, wherein the at least one surfactant is selected from the group consisting of non-phospholipid surfactants, polyoxyethylene derivatives of sorbitan fatty acid esters, nonphenol polyethylene glycol ethers, sorbitan esters, glycerol esters, glycerin monostearate, polyethylene glycol esters, polyethylene glycol stearate, poloxamers, block polymers, acrylic polymers, ethoxylated fatty esters, ethoxylated alcohols, ethoxylated fatty acids, monoglycerides, silicon based surfactants, polysorbates, Poly(oxy-1,2-ethanediyl, α-(4-nonylphenol)-omega-hydroxy, branched [molecular weight average 1980]), and Tergitol NP-70.

9. The formulation of claim 1, wherein the individual surfactant molecules are free of cross-linkages.

10. The formulation of claim 1, wherein the water is present in Phosphate Buffered Saline (PBS).

11. The formulation of claim 1, further comprising a solvent.

12. The formulations of claim 11, wherein the solvent is selected from the group consisting of to C₁-C₁₂ alcohols, isopropyl myristate, triacetin, N-methylpyrrolidinone, aliphatic alcohols, aromatic alcohols, polyethylene glycols, ethanol, propylene glycol, dimethyl sulfoxide, dimethyl acetamide, ethoxydiglycol, and any combination thereof.

13. The formulation of claim 1, wherein the formulation further comprises an antioxidant, a microbial preservative, a viscosity promoting agent, or any combination thereof.

14. The formulation of claim 13, wherein:
(a) the antioxidant is selected from the group consisting of sodium metabisulfite, ascorbic acid, and alpha tocopherol;
(b) the microbial preservative is selected from the group consisting of sorbic acid, methyl paraben, ethyl paraben, propyl paraben, chlorobutanol, and benzalkonium chloride;
(c) the viscosity promoting agent is selected from the group consisting of hydroxypropylmethylcellulose, ethylcellulose and methylcellulose; or
(d) any combination thereof.

15. The formulation of claim 1 comprising:
(a) about 0.0001 wt. % to about 15 wt. % of at least one hormone, or about 0.001 wt. % to about 5.0 wt. % of at least one hormone;
(b) about 0.01 wt. % to about 50 wt. % of at least one surfactant, or about 3.0 wt. % to about 10.0 wt. % of at least one surfactant;
(c) about 10 wt. % to about 90 wt. % of water, or about 40 wt. % to about 60 wt. % of water; and
(d) about 10 wt. % to about 85 wt. % of squalene, squalane, super refined soybean oil, and/or a medium chain triglyceride, or about 40 wt. % to about 65 wt. % of squalene, squalane, super refined soybean oil, and/or a medium chain triglyceride, or any combination thereof.

16. A dilution of the formulation of claim 1.

17. The formulation of claim 1, wherein the droplets have an average diameter selected from the group consisting of less than about 10 microns to about 1 nm.

18. The formulation of claim 1, wherein:
(a) the formulation is thermostable; and/or
(b) the formulation is stable when exposed to a temperature of about 50° C.

19. The formulation of claim 1, wherein:
(a) the composition is in a dosage form selected from the group consisting of liquids, ointments, creams, oils, emulsions, lotions, gels, sprays, aerosols, pastes, foams, patch in the form of an article or carrier, such as a bandage, insert, pessary, powder, talc or other solid;
(b) the composition is a controlled release formulation, sustained release formulation, immediate release formulation, or any combination thereof; or
(c) any combination thereof.

20. The formulation of claim 1, wherein the formulation further comprises at least one non-hormone active agent.

21. The formulation of claim 20, wherein the non-hormone agent is selected from the group consisting of a drug substance, a non-drug substance, antiinfective agent, antifungal agent, antiviral agent, NSAID, topical steroid, topical anesthetic agent, skin lightening agent, tanning agent, skin conditioning agent, skin protectant, emollient, humectant, botulinum toxin type A, a retinoid, isotretinoin, alitretinoin, etretinate, acitretin, tazarotene, bexarotene, alpha hydroxy acids, beta hydroxy acids, poly hydroxy acids, hydroxyl acids, kinetin, coenzyme Q10, copper peptides, tea extracts, antioxidants, glutathione, melatonin, tocopherols, α-tocopherol, tocotrienols, lipoic acid, uric acid, carotenes, ubiquinone, thioredoxin, Polyphenolic antioxidants, carotenoids, and any mixture thereof.

22. A method of treating, preventing, minimizing, and/or diminishing a dermatological condition comprising topically administering a hormonal nano formulation to a subject, wherein the formulation is as recited in claim 21.

23. The method of claim 22, wherein the dermatological condition is selected from the group consisting of:
(a) fine to moderate wrinkles,
(b) liver spots or age spots (lentigines or solar lentigines),
(c) uneven skin tone and/or texture,
(d) sun-damaged skin or photodamaged skin (particularly UV radiation-induced oxidative stress),
(e) blemishes,
(f) hyperpigmented skin,
(g) increased skin thickness,
(h) dry skin,
(i) loss of skin elasticity and collagen content,
(j) melasmas (atypical pigmentation or hyper-pigmentation of the skin),
(k) skin clarity and/or radiance,
(l) skin smoothness and/or softness,
(m) scars,
(n) pore size,
(o) hydration,
(p) skin smoothness,
(q) skin tightness,
(r) herpes lesion;
(s) skin wounds;
(t) skin infection; and
(u) any combination thereof.

24. The method of claim 22, wherein the formulation is topically applied:
(a) in a single administration;
(b) for at least once a week, at least twice a week, at least once a day, at least twice a day, multiple times daily, multiple times weekly, biweekly, at least once a month, or any combination thereof;
(c) for a period of time selected from the group consisting of about one week, about two weeks, about three weeks, about one month, or up to about 5 years; or
(d) any combination thereof.

25. A method of treating a subject in need with a hormonal nano formulation, wherein the subject has a dermatological condition needing treatment, and the formulation comprises:
(a) at least one hormone;
(b) at least one surfactant;
(c) water; and
(d) squalene, squalane, super refined soybean oil, a medium chain triglyceride, or any combination thereof,
wherein the formulation comprises droplets having an average diameter of less than about 10 microns.

26. The method of claim 25, wherein the subject has diabetes and the dermatological condition is a wound.

27. The method of claim 26, wherein the formulation further comprises an antiinfective agent.

28. The method of claim 25, wherein the formulation comprises a topical steroid, and wherein the method results in lowing the incidence of itching, rash and dermatitis associated with the dermatological condition.

29. The method of claim 25, wherein the formulation comprises a topical antifungal agent, and wherein the method results in relieving complications of eczema, psoriasis, and/or other dermatological maladies.

30. The method of claim 25, wherein the formulation comprises a topical NSAID, and wherein the method results in lowering or countering local pain, inflammation and/or discomfort associated with the dermatological condition.

31. The method of claim 25, wherein the formulation comprises a topical anesthetic, and wherein the method results in easing the pain and/or discomfort associated with the dermatological condition.

32. The method of claim 25, wherein the formulation comprises a topical antiviral agent, and wherein the method results in decreasing symptoms and/or complication associated with genital and/or oral herpes infections.
